SEQUENCE LISTING (37 sequences)

SEQ ID NO: 1 - DNA, 951 bp, Rotavirus strain SA11
atggctgagctagcttgcttttgctatcctcatttggagaatgatagctataaatttattccttttaataatttagctattaaagctatgctgacagctaaagtagacaaaaaggacatggataagttttatgattcaattatttatggaatagcaccgcctcctcaatttaagaaacggtataatactaatgataattcaagaggcatgaattttgaaacaattatgtttactaaggtggctatgttgatatgtgaagctctaaattcattgaaagtgacgcaagcaaacgtctctaatgtattatcacgagtagtatcaataaggcatttagaaaatttggtgatacgtaaagaaaatccacaggatattctatttcattcaaaagatttacttttgaaatcaacactgatcgctattggacagtctaaagaaattgaaactacaataactgcagaaggaggagaaattgtatttcaaaacgctgccttcaccatgtggaaactaacttatttagaacatcaattgatgccaattctggatcagaattttattgaatataaagttacattgaacgaagataaaccaatttcagatgttcatgttaaagaattagtcgctgaacttcgatggcaatataacaagtttgctgtaatcacacatggtaagggtcattatagaattgtaaagtattcatcagttgctaatcacgctgacagagtatatgcaacttccaagagtaatgttaaaactggagttaataatgattttaacctacttgatcaaagaattatttggcaaaactggtatgcatttacatcatcaatgaaacagggtaatacacttgacgtgtgtaaaaggttgcttttccaaaaactgaaaccagaaaaaaatccatttaaagggctgtcaacggatagaaaaatggacgaagtttctcaagttggcgtt

SEQ ID NO: 2 - protein, 317 aa, Rotavirus strain SA11
MAELACFCYPHLENDSYKFIPFNNLAIKAMLTAKVDKKDMDKFYDSIIYGIAPPPQFKKRYNTNDNSRGMNFETIMFTKVAMLICEALNSLKVTQANVSNVLSRVVSIRHLENLVIRKENPQDILFHSKDLLLKSTLIAIGQSKEIETTITAEGGEIVFQNAAFTMWKLTYLEHQLMPILDQNFIEYKVTLNEDKPISDVHVKELVAELRWQYNKFAVITHGKGHYRIVKYSSVANHADRVYATSKSNVKTGVNNDFNLLDQRIIWQNWYAFTSSMKQGNTLDVCKRLLFQKLKPEKNPFKGLSTDRKMDEVSQVGV

SEQ ID NO: 3 - DNA, 936 bp, Rotovirus strain Bristol
atggccgagctagcctgtttcgtttccttctcactgactgaagataaagtagtatggtatccaattaataaaaaagcagtacaaaccatgttgtgtgctaaagtggaaaaagatcagaggagtaattattatgatactatactatatggtgttgcaccaccaccggagtttagaaatagatttaaaacaaatgaaagatatggcttagactatgaatcagatcaatataccgaattagttaatctgttagcagatacattgaatatggtgtctatgccaactgaaaaatttcaatttgacatagttaaaacagtagtgcaagttaggcatcttgaaaatttgctttgcagaataaaagatgttaatgatattttgaatgcaaatgtgaaattaagagtaaaagcagttatgattgcatgcaatttagtaaatgaaactgaaactactccattgacagagagtaatgatattgtatatcaggattcatattttacgattacaaaattagattattccaatcataaattacttccattaatggctgatgaatataaaattacaattaacacaaaaactgatattccagatcgtaatcaaactgcatttgcagcttatattagatataattttaataaatttgcggcaatatctcatggtaaacggcattggaggcttgttcttcattctcagttgatgtctcatgcagaaagattagatagaaaaattaagtcggataaaaaacatggtagacaattctcttatgatgatggagatatggctttcgtacatccaggatggaaaacgtgtattggtcaattatgtggtggtactacatttgaagttgctaaaacatcactatacagcataaaaccatcaaaaactgtgcgcacagcgacaaataaaattgaaagtgacttaatttctatggttggaaat

SEQ ID NO: 4 - protein, 312 aa, Rotovirus strain Bristol
MAELACFVSFSLTEDKVVWYPINKKAVQTMLCAKVEKDQRSNYYDTILYGVAPPPEFRNRFKTNERYGLDYESDQYTELVNLLADTLNMVSMPTEKFQFDIVKTVVQVRHLENLLCRIKDVNDILNANVKLRVKAVMIACNLVNETETTPLTESNDIVYQDSYFTITKLDYSNHKLLPLMADEYKITINTKTDIPDRNQTAFAAYIRYNFNKFAAISHGKRHWRLVLHSQLMSHAERLDRKIKSDKKHGRQFSYDDGDMAFVHPGWKTCIGQLCGGTTFEVAKTSLYSIKPSKTVRTATNKIESDLISMVGN

SEQ ID NO: 5 - DNA, 57 bp, Rotavirus strain RRV
gaaggaaaactatctgaagaatatgagcttctgatctcggactctgaagatgacgat

SEQ ID NO: 6 - protein, 19 aa, Rotavirus strain RRV
EGKLSEEYELLISDSEDDD

SEQ ID NO: 7 - DNA, 60 bp, Rotavirus strain SA11
tctggaactttaactgaagaatttgagctactgatctccaactcagaagatgacaatgag

SEQ ID NO: 8 - protein, 20 aa, Rotavirus strain SA11
SGTLTEEFELLISNSEDDNE

SEQ ID NO: 9 - DNA, 495 bp, Rotavirus strain RRV
actgtagaaccggtacttgatggtccttatcaaccaacaacgttcaatccaccagtagattattggatgctattagcacctacagcagctggagtagtagtagaaggtactaataatacagaccgatggctagctacaattttagttgagccaaacgtaacatcagaaaccagaagttatacgctatttggaacgcaagagcaaattacaatagctaatgcttcccaaacacaatggaaatttattgatgtcgttaaaactacacaaaatggaagctattcacaatacggaccattacaatctactccaaaactctatgccgtgatgaaacataatggtaaaatttatacatataatggagaaactccgaatgtgaccactaagtactactcaactacaaattatgattcagtaaacatgacagcattttgtgacttttatattatacctagagaagaagaatcaacatgtaccgagtacattaataacgggtta

SEQ ID NO: 10 - protein, 165 aa, Rotavirus strain RRV
TVEPVLDGPYQPTTFNPPVDYWMLLAPTAAGVVVEGTNNTDRWLATILVEPNVTSETRSYTLFGTQEQITIANASQTQWKFIDVVKTTQNGSYSQYGPLQSTPKLYAVMKHNGKIYTYNGETPNVTTKYYSTTNYDSVNMTAFCDFYIIPREEESTCTEYINNGL

SEQ ID NO: 11 - DNA, 18 bp, Artificial
  NT Sequence Encoding His Tag
catcaccatcaccatcac

SEQ ID NO: 12 - protein, 6 aa, Artificial
  Hexa-His Tag
HHHHHH

SEQ ID NO: 13 - DNA, 12 bp, Artificial
  NT Sequence Encoding GPGP Linker
ggaccaggcccg

SEQ ID NO: 14 - protein, 4 aa, Artificial
  GPGP Linker
GPGP

SEQ ID NO: 15 - DNA, 1047 bp, Artificial
  NSP2-RRV.NSP1(C19) Fusion Protein
atggctgagctagcttgcttttgctatcctcatttggagaatgatagctataaatttattccttttaataatttagctattaaagctatgctgacagctaaagtagacaaaaaggacatggataagttttatgattcaattatttatggaatagcaccgcctcctcaatttaagaaacggtataatactaatgataattcaagaggcatgaattttgaaacaattatgtttactaaggtggctatgttgatatgtgaagctctaaattcattgaaagtgacgcaagcaaacgtctctaatgtattatcacgagtagtatcaataaggcatttagaaaatttggtgatacgtaaagaaaatccacaggatattctatttcattcaaaagatttacttttgaaatcaacactgattgctattggacagtctaaagaaattgaaactacaataactgtagaaggaggagaaattgtatttcaaaacgctgccttcaccatgtggaaactaacttatttagaacatcaattgatgccaattctggatcagaattttattgaatataaagttacattgaacgaagataaaccaatttcagatattcatgttaaagaattagtcgctgaacttcgatggcaatataacaagtttgctgtaatcacacatggtaagggtcattatagaattgtaaagtattcatcagttgctaatcacgctgacagagtatatgcaactttcaagagtaatgttaaaactggagttaataatgattttaacctacttgatcaaagaattatttggcaaaactggtatgcatttacatcatcaatgaaacagggtaatacacttgacgtgtgtaaaaggttgcttttccaaaaaatgaaaccagaaaaaaatccatttaaagggctgtcaacggatagaaaaatggacgaagtttctcaagttggcgttagatctcatcaccatcaccatcacggaccaggcccggaaggaaaactatctgaagaatatgagcttctgatctcggactctgaagatgacgattaa

SEQ ID NO: 16 - protein, 348 aa, Artificial
  Synthetic Construct
MAELACFCYPHLENDSYKFIPFNNLAIKAMLTAKVDKKDMDKFYDSIIYGIAPPPQFKKRYNTNDNSRGMNFETIMFTKVAMLICEALNSLKVTQANVSNVLSRVVSIRHLENLVIRKENPQDILFHSKDLLLKSTLIAIGQSKEIETTITVEGGEIVFQNAAFTMWKLTYLEHQLMPILDQNFIEYKVTLNEDKPISDIHVKELVAELRWQYNKFAVITHGKGHYRIVKYSSVANHADRVYATFKSNVKTGVNNDFNLLDQRIIWQNWYAFTSSMKQGNTLDVCKRLLFQKMKPEKNPFKGLSTDRKMDEVSQVGVRSHHHHHHGPGPEGKLSEEYELLISDSEDDD

SEQ ID NO: 17 - DNA, 1050 bp, Artificial
  NSP2-SA11.NSP1(C19) Fusion Protein
atggctgagctagcttgcttttgctatcctcatttggagaatgatagctataaatttattccttttaataatttagctattaaagctatgctgacagctaaagtagacaaaaaggacatggataagttttatgattcaattatttatggaatagcaccgcctcctcaatttaagaaacggtataatactaatgataattcaagaggcatgaattttgaaacaattatgtttactaaggtggctatgttgatatgtgaagctctaaattcattgaaagtgacgcaagcaaacgtctctaatgtattatcacgagtagtatcaataaggcatttagaaaatttggtgatacgtaaagaaaatccacaggatattctatttcattcaaaagatttacttttgaaatcaacactgattgctattggacagtctaaagaaattgaaactacaataactgtagaaggaggagaaattgtatttcaaaacgctgccttcaccatgtggaaactaacttatttagaacatcaattgatgccaattctggatcagaattttattgaatataaagttacattgaacgaagataaaccaatttcagatattcatgttaaagaattagtcgctgaacttcgatggcaatataacaagtttgctgtaatcacacatggtaagggtcattatagaattgtaaagtattcatcagttgctaatcacgctgacagagtatatgcaactttcaagagtaatgttaaaactggagttaataatgattttaacctacttgatcaaagaattatttggcaaaactggtatgcatttacatcatcaatgaaacagggtaatacacttgacgtgtgtaaaaggttgcttttccaaaaaatgaaaccagaaaaaaatccatttaaagggctgtcaacggatagaaaaatggacgaagtttctcaagttggcgttagatctcatcaccatcaccatcacggaccaggcccgtctggaactttaactgaagaatttgagctactgatctccaactcagaagatgacaatgagtaa

SEQ ID NO: 18 - protein, 349 aa, Artificial
  Synthetic Construct
MAELACFCYPHLENDSYKFIPFNNLAIKAMLTAKVDKKDMDKFYDSIIYGIAPPPQFKKRYNTNDNSRGMNFETIMFTKVAMLICEALNSLKVTQANVSNVLSRVVSIRHLENLVIRKENPQDILFHSKDLLLKSTLIAIGQSKEIETTITVEGGEIVFQNAAFTMWKLTYLEHQLMPILDQNFIEYKVTLNEDKPISDIHVKELVAELRWQYNKFAVITHGKGHYRIVKYSSVANHADRVYATFKSNVKTGVNNDFNLLDQRIIWQNWYAFTSSMKQGNTLDVCKRLLFQKMKPEKNPFKGLSTDRKMDEVSQVGVRSHHHHHHGPGPSGTLTEEFELLISNSEDDNE

SEQ ID NO: 19 - DNA, 1539 bp, Artificial
  NSP2-GPGP-RRV.V8 Fusion Protein
atggctgagctagcttgcttttgctatcctcatttggagaatgatagctataaatttattccttttaataatttagctattaaagctatgctgacagctaaagtagacaaaaaggacatggataagttttatgattcaattatttatggaatagcaccgcctcctcaatttaagaaacggtataatactaatgataattcaagaggcatgaattttgaaacaattatgtttactaaggtggctatgttgatatgtgaagctctaaattcattgaaagtgacgcaagcaaacgtctctaatgtattatcacgagtagtatcaataaggcatttagaaaatttggtgatacgtaaagaaaatccacaggatattctatttcattcaaaagatttacttttgaaatcaacactgatcgctattggacagtctaaagaaattgaaactacaataactgcagaaggaggagaaattgtatttcaaaacgctgccttcaccatgtggaaactaacttatttagaacatcaattgatgccaattctggatcagaattttattgaatataaagttacattgaacgaagataaaccaatttcagatgttcatgttaaagaattagtcgctgaacttcgatggcaatataacaagtttgctgtaatcacacatggtaagggtcattatagaattgtaaagtattcatcagttgctaatcacgctgacagagtatatgcaacttccaagagtaatgttaaaactggagttaataatgattttaacctacttgatcaaagaattatttggcaaaactggtatgcatttacatcatcaatgaaacagggtaatacacttgacgtgtgtaaaaggttgcttttccaaaaactgaaaccagaaaaaaatccatttaaagggctgtcaacggatagaaaaatggacgaagtttctcaagttggcgttagatctcatcaccatcaccatcacggaccaggcccgagcggccgcagcactgtagaaccggtacttgatggtccttatcaaccaacaacgttcaatccaccagtagattattggatgctattagcacctacagcagctggagtagtagtagaaggtactaataatacagaccgatggctagctacaattttagttgagccaaacgtaacatcagaaaccagaagttatacgctatttggaacgcaagagcaaattacaatagctaatgcttcccaaacacaatggaaatttattgatgtcgttaaaactacacaaaatggaagctattcacaatacggaccattacaatctactccaaaactctatgccgtgatgaaacataatggtaaaatttatacatataatggagaaactccgaatgtgaccactaagtactactcaactacaaattatgattcagtaaacatgacagcattttgtgacttttatattatacctagagaagaagaatcaacatgtaccgagtacattaataacgggttaagcggccgcggaccaggcccgtctcatcaccatcaccatcactaa

SEQ ID NO: 20 - protein, 512 aa, Artificial
  Synthetic Construct
MAELACFCYPHLENDSYKFIPFNNLAIKAMLTAKVDKKDMDKFYDSIIYGIAPPPQFKKRYNTNDNSRGMNFETIMFTKVAMLICEALNSLKVTQANVSNVLSRVVSIRHLENLVIRKENPQDILFHSKDLLLKSTLIAIGQSKEIETTITAEGGEIVFQNAAFTMWKLTYLEHQLMPILDQNFIEYKVTLNEDKPISDVHVKELVAELRWQYNKFAVITHGKGHYRIVKYSSVANHADRVYATSKSNVKTGVNNDFNLLDQRIIWQNWYAFTSSMKQGNTLDVCKRLLFQKLKPEKNPFKGLSTDRKMDEVSQVGVRSHHHHHHGPGPSGRSTVEPVLDGPYQPTTFNPPVDYWMLLAPTAAGVVVEGTNNTDRWLATILVEPNVTSETRSYTLFGTQEQITIANASQTQWKFIDVVKTTQNGSYSQYGPLQSTPKLYAVMKHNGKIYTYNGETPNVTTKYYSTTNYDSVNMTAFCDFYIIPREEESTCTEYINNGLSGRGPGPSHHHHHH

SEQ ID NO: 21 - DNA, 1506 bp, Artificial
  NSP2-GGS-RRV.VP8 Fusion Protein
atggctgagctagcttgcttttgctatcctcatttggagaatgatagctataaatttattccttttaataatttagctattaaagctatgctgacagctaaagtagacaaaaaggacatggataagttttatgattcaattatttatggaatagcaccgcctcctcaatttaagaaacggtataatactaatgataattcaagaggcatgaattttgaaacaattatgtttactaaggtggctatgttgatatgtgaagctctaaattcattgaaagtgacgcaagcaaacgtctctaatgtattatcacgagtagtatcaataaggcatttagaaaatttggtgatacgtaaagaaaatccacaggatattctatttcattcaaaagatttacttttgaaatcaacactgatcgctattggacagtctaaagaaattgaaactacaataactgcagaaggaggagaaattgtatttcaaaacgctgccttcaccatgtggaaactaacttatttagaacatcaattgatgccaattctggatcagaattttattgaatataaagttacattgaacgaagataaaccaatttcagatgttcatgttaaagaattagtcgctgaacttcgatggcaatataacaagtttgctgtaatcacacatggtaagggtcattatagaattgtaaagtattcatcagttgctaatcacgctgacagagtatatgcaactttcaagagtaatgttaaaactggagttaataatgattttaacctacttgatcaaagaattatttggcaaaactggtatgcatttacatcatcaatgaaacagggtaatacacttgacgtgtgtaaaaggttgcttttccaaaaactgaaaccagaaaaaaatccatttaaagggctgtcaacggatagaaaaatggacgaagtttctcaagttggcgttggtggctcagaaccggtacttgatggtccttatcaaccaacaacgttcaatccaccagtagattattggatgctattagcacctacagcagctggagtagtagtagaaggtactaataatacagaccgatggctagctacaattttagttgagccaaacgtaacatcagaaaccagaagttatacgctatttggaacgcaagagcaaattacaatagctaatgcttcccaaacacaatggaaatttattgatgtcgttaaaactacacaaaatggaagctattcacaatacggaccattacaatctactccaaaactctatgccgtgatgaaacataatggtaaaatttatacatataatggagaaactccgaatgtgaccactaagtactactcaactacaaattatgattcagtaaacatgacagcattttgtgacttttatattatacctagagaagaagaatcaacatgtaccgagtacattaataacgggttaggtggctcaggtggatccttagtacctagaggaagccatcaccatcaccatcactaa

SEQ ID NO: 22 - protein, 501 aa, Artificial
  Synthetic Construct
MAELACFCYPHLENDSYKFIPFNNLAIKAMLTAKVDKKDMDKFYDSIIYGIAPPPQFKKRYNTNDNSRGMNFETIMFTKVAMLICEALNSLKVTQANVSNVLSRVVSIRHLENLVIRKENPQDILFHSKDLLLKSTLIAIGQSKEIETTITAEGGEIVFQNAAFTMWKLTYLEHQLMPILDQNFIEYKVTLNEDKPISDVHVKELVAELRWQYNKFAVITHGKGHYRIVKYSSVANHADRVYATFKSNVKTGVNNDFNLLDQRIIWQNWYAFTSSMKQGNTLDVCKRLLFQKLKPEKNPFKGLSTDRKMDEVSQVGVGGSEPVLDGPYQPTTFNPPVDYWMLLAPTAAGVVVEGTNNTDRWLATILVEPNVTSETRSYTLFGTQEQITIANASQTQWKFIDVVKTTQNGSYSQYGPLQSTPKLYAVMKHNGKIYTYNGETPNVTTKYYSTTNYDSVNMTAFCDFYIIPREEESTCTEYINNGLGGSGGSLVPRGSHHHHHH

SEQ ID NO: 23 - DNA, 1017 bp, Artificial
  NSP2-HHHHHH Fusion Protein
atggccgagctagcctgtttcgtttccttctcactgactgaagataaagtagtatggtatccaattaataaaaaagcagtacaaaccatgttgtgtgctaaagtggaaaaagatcagaggagtaattattatgatactatactatatggtgttgcaccaccaccggagtttagaaatagatttaaaacaaatgaaagatatggcttagactatgaatcagatcaatataccgaattagttaatctgttagcagatacattgaatatggtgtctatgccaactgaaaaatttcaatttgacatagttaaaacagtagtgcaagttaggcatcttgaaaatttgctttgcagaataaaagatgttaatgatattttgaatgcaaatgtgaaattaagagtaaaagcagttatgattgcatgcaatttagtaaatgaaactgaaactactccattgacagagagtaatgatattgtatatcaggattcatattttacgattacaaaattagattattccaatcataaattacttccattaatggctgatgaatataaaattacaattaacacaaaaactgatattccagatcgtaatcaaactgcatttgcagcttatattagatataattttaataaatttgcggcaatatctcatggtaaacggcattggaggcttgttcttcattctcagttgatgtctcatgcagaaagattagatagaaaaattaagtcggataaaaaacatggtagacaattctcttatgatgatggagatatggctttcgtacatccaggatggaaaacgtgtattggtcaattatgtggtggtactacatttgaagttgctaaaacatcactatacagcataaaaccatcaaaaactgtgcgcacagcgacaaataaaattgaaagtgacttaatttctatggttggaaatagatctcatcaccatcaccatcacggaccaggcccgagcggccgcggaccaggcccgtctcatcaccatcaccatcactaa

SEQ ID NO: 24 - protein, 338 aa, Artificial
  Synthetic Construct
MAELACFVSFSLTEDKVVWYPINKKAVQTMLCAKVEKDQRSNYYDTILYGVAPPPEFRNRFKTNERYGLDYESDQYTELVNLLADTLNMVSMPTEKFQFDIVKTVVQVRHLENLLCRIKDVNDILNANVKLRVKAVMIACNLVNETETTPLTESNDIVYQDSYFTITKLDYSNHKLLPLMADEYKITINTKTDIPDRNQTAFAAYIRYNFNKFAAISHGKRHWRLVLHSQLMSHAERLDRKIKSDKKHGRQFSYDDGDMAFVHPGWKTCIGQLCGGTTFEVAKTSLYSIKPSKTVRTATNKIESDLISMVGNRSHHHHHHGPGPSGRGPGPSHHHHHH

SEQ ID NO: 25 - DNA, 21 bp, Artificial
  G8 Forward Primer
ccgaaaccatggctgagctag

SEQ ID NO: 26 - DNA, 26 bp, Artificial
  G8 Reverse Primer
cggagatctacgccaacttgagaaac

SEQ ID NO: 27 - DNA, 60 bp, Artificial
  RRV C19 Forward Primer
cttctgatctcggactctgaagatgacgattaagcttaattagctgagcttggactcctg

SEQ ID NO: 28 - DNA, 69 bp, Artificial
  RRV C19 Reverse Primer
ctcatattcttcagatagttttccttccgggcctggtccgtgatggtgatggtgatgagatctaacgcc

SEQ ID NO: 29 - DNA, 63 bp, Artificial
  SA11 C19 Forward Primer
ctactgatctccaactcagaagatgacaatgagtaagcttaattagctgagcttggactcctg

SEQ ID NO: 30 - DNA, 69 bp, Artificial
  SA11 C19 Reverse Primer
ctcaaattcttcagttaaagttccagacgggcctggtccgtgatggtgatggtgatgagatctaacgcc

SEQ ID NO: 31 - DNA, 16 bp, Artificial
  Primer
ctggtgccatggccga

SEQ ID NO: 32 - DNA, 27 bp, Artificial
  Primer
cactacaagatctatttccaaccatag

SEQ ID NO: 33 - DNA, 32 bp, Artificial
  Primer
gagcggccgctcgggcctggtccgtgatggtg

SEQ ID NO: 34 - DNA, 85 bp, Artificial
  Primer
gaagcggccgcggaccaggcccgtctcatcaccatcaccatcactaagcttaattagctgagcttggactcctgttgatagatcc

SEQ ID NO: 35 - DNA, 47 bp, Artificial
  Primer
ccgcggccgctcgggcctggtccgtgatggtgatggtgatgagatct

SEQ ID NO: 36 - DNA, 50 bp, Artificial
  Primer
ccgcggccgctcgggcctggtccgtgatggtgatggtgatgatgagatct

SEQ ID NO: 37 - DNA, 3431 bp, Artificial
  Vector
ctcgagaaatcataaaaaatttatttgctttgtgagcggataacaattataatagattcaattgtgagcggataacaatttcacacagaattcattaaagaggagaaattaaccatgggaggatccagatctcatcaccatcaccatcactaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtctgtcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagctgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

(19) United States
(12) Patent Application Publication
(10) Pub. No.: US 2006/0122368 A1
(43) Pub. Date: Jun. 8, 2006

(54) Antigen delivery platform

(75) Inventors: John T. Patton, Fairfax, VA (US); Zenobia F. Taraporewala, Silver Spring, MD (US)

Correspondence Address: KLARQUIST SPARKMAN, LLP, 121 S.W. SALMON STREET, SUITE #1600, PORTLAND, OR, 97204-2988, US

(73) Assignee: The Government of the U.S.A. as represented by the Secretary of the Dept. of Health & Human Services

(21) Appl. No.: 11/293,654

(22) Filed: Dec. 2, 2005

Related U.S. Application Data
(60) Provisional application No. 60/633,036, filed on Dec. 3, 2004.

(51) Int. Cl. C07K 14/00; C07K 16/00; C07K 1/00; A61K 38/00; C07K 17/00; C07K 2/00; C07K 4/00; C07K 5/00; C07K 7/00

(52) U.S. Cl. 530/300; 530/350

(57) ABSTRACT

Polynucleotides encoding fusion proteins suitable as an antigen delivery platform are provided. Pharmaceutical compositions including the fusion proteins and methods for their use are also provided.

### CROSS REFERENCE TO RELATED APPLICATION

This application claims priority to and benefit of U.S. provisional application no. 60/633,036, filed Dec. 3, 2004, the specification of which is incorporated herein in its entirety for all purposes.

### FIELD

This disclosure relates to the field of vaccines. Specifically, this disclosure relates to Rotavirus NSP2 fusion proteins and their use as an antigen delivery platform.

### BACKGROUND

Rotaviruses are a significant cause of diarrheal disease in humans and other animals. The rotavirion is a triple-layer icosahedron formed by the outer-layer proteins, VP7 and VP4, the middle-layer protein, VP6, and the core matrix protein, VP2. Closely associated with the VP2 matrix are the RNA-dependent RNA polymerase, VP1, and the multifunctional capping enzyme, VP3. During the replication cycle, viral mRNAs serve as templates for the synthesis of minus-strand RNA to form the dsRNA genome. Synthesis of the genome segments occurs concurrently with the packaging of the mRNAs into core-like replication intermediates (RIs) consisting of not only the structural but also the nonstructural proteins. The formation of RIs and the replication of the dsRNA genome occur in large cytoplasmic inclusions (viroplasms) that form in infected cells.

The roles of the nonstructural proteins in RNA packaging and replication are not fully understood. However, studies of a mutant strain of virus with a temperature sensitive (ts) lesion in NSP2, indicate that this protein plays an important role in these processes.

NSP2 (mass, 35 kDa) is a nonspecific ssRNA-binding protein that has been shown to assemble into multimers, which have been detected in infected cells (Kattoura et al., Virology 191:698-708, 1992; Taraporewala et al., J. Virol. 73:9934-9943, 1999). Hydrodynamic and thermodynamic studies indicate that these multimers are highly stable octamers, formed by very specific and strong self-assembly resulting from the interaction between two NSP2 tetramers. These octamers are the oligomeric state relevant for RNA binding of the protein as well as NTPase activity (Schuck et al., J. Biol. Chem. 276:9679-9687, 2001). The octamers bind to ssRNA in a cooperative fashion, giving rise to higher-order complexes composed of a single RNA molecule and multiple copies of the octamer (Taraporewla and Patton, J. Virol. 75:4519-4527, 2001). NSP2 octamers also possess a nucleotide- and Mg2+-independent helix-destabilization activity (Taraporewala et al., J. Virol. 73:9934-9943, 1999), a function that has been suggested to remove RNA-RNA duplexes in viral mRNAs that may inhibit RNA packaging and replication (Qiao et al., J. Virol. 69:5502-5505, 1995). Besides RNA-binding activity, NSP2 octamers display an associated Mg2+-dependent NTPase activity that may provide energy for packaging. In the presence of Mg2+ and nucleotides, NSP2 octamers undergo a conformational change into a more condensed form, whereas Mg2+ alone promotes the dissociation of the octamers into tetramers (Schuck et al., J. Biol. Chem. 276:9679-9687, 2001).

The NSP2 multimer possesses a number of beneficial attributes that are disclosed herein, and which form the basis of a versatile molecular platform for the assembly and delivery of antigenic epitopes.

### SUMMARY

The present disclosure concerns an epitope mounting platform that is suitable for the production and delivery of a wide variety of antigens. The antigen delivery platform is based on the beneficial attributes of NSP2 fusion proteins. Methods for producing and using NSP2 based antigen delivery platforms are also disclosed herein.

An antigen delivery platform suitable for production and administration of a wide spectrum of antigenic epitopes is described herein. The antigen delivery platform is based on a fusion protein which includes a) a self-aggregating polypeptide component that induces the formation of a stable multimeric ring structure; b) a linear linking peptide that preserves the secondary and tertiary structure of the self-aggregating polypeptide component within the fusion protein multimer; and, c) an antigenic polypeptide to which an immune response is desirable.

In an embodiment, the self-aggregating polypeptide is a viral NSP2 polypeptide that induces formation of fusion protein multimers in which the antigenic polypeptide is exposed on the surface of a stable ring structure making it available to the immune system to stimulate a specific immune response against the antigenic epitope. The fusion protein ring structures can be produced in host cells with high efficiency and can be recovered under non-denaturing conditions yielding an antigen delivery platform that is stable under a wide range of pH, temperature and ionic conditions.

Polynucleotides encoding the self-aggregating fusion proteins are also described, as are pharmaceutical compositions, including vaccines, suitable for prophylactic and therapeutic administration, and methods for their use.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic representation of the structure of a monomeric NSP2 fusion protein illustrating the distinct constituent elements in an N-terminal to C-terminal direction. The linear linking peptide is shown in black. The antigenic polypeptide portion of the fusion protein is indicated by diagonal stripes.

FIG. 2 is a graphical model representing the 3-dimensional structure of an NSP2/VP8 fusion protein ring structure. The left panel illustrates the octameric ring structure formed by isolated NSP2 protein. The right panel illustrates the arrangement of the VP8 epitope at the perimeter of the octamer.

FIG. 3 is an image depicting a Coomassie Blue stained polyacrylamide gel. NSP2-NSP1.C19 fusion proteins were expressed and purified. From the left, lane 1 contains molecular weight markers. Lanes 2-5 contain increasing concentrations of a bovine serum albumin (“BSA”) protein standard. Lane 7 contains NSP2 protein and lanes 8 and 9 are NSP2-NSP1.C19 fusion proteins, wherein the fusion partner, NSP1 C19, is derived from the SA11 and RRV viruses, respectively.

FIGS. 4A and B are images illustrating a Coomassie Blue stained polyacrylamide gel and a Western blot analysis, respectively. A. Left panel illustrates expression and purification of an NSP2-VP8 fusion protein joined by an intervening GPGP linking peptide (lane M: molecular weight standards; lane 1: lysate from uninduced bacteria; lane 2: lysate from bacteria induced with 1 mM IPTG; lane 3: soluble fraction of the induced bacterial lysate; lane 4: flow through fraction following NTA-agarose column chromatography; lane 5: eluate after dialysis of nickel-bound fraction). Right panel illustrates expression and purification of an NSP2-VP8 fusion protein joined by an intervening GGS linking peptide (lane M: molecular weight standards; lane 1: lysate from uninduced bacteria; lane 2: lysate from bacteria induced with ImM IPTG; lane 3: soluble fraction of the induced bacterial lysate; lane 4: flow through fraction following NTA-agarose column chromatography; lanes 5 and 6: eluate after dialysis of nickel-bound fraction). B. illustrates detection of the VP8 antigenic epitope (VP4) by serum antibodies from guinea pigs immunized with an NSP2-VP8 (GPGP) fusion protein (lane 1: post-immunization serum; lane 2: pre-immunization serum; lane 3: guinea pig hyperimmune sera raised against the D×RRV virus).

FIGS. 5A and B are images of a Coomassie Blue stained polyacrylamide gel and a Western blot, respectively. A. illustrates expression and purification of the SA11 NSP2 octamer (left panel) and the Bristol NSP2 octamer (right panel). B. demonstrates the lack of antigenic cross-reactivity between SA11- and Bristol-derived NSP2 octamers. Lanes numbered in ascending order 1-6 indicate eluate fraction.

FIG. 6 is an image of a western blot analysis illustrating the presence of serum antibodies against the NSP1.C19 antigenic polypeptide in guinea pigs immunized with an NSP2-NSP1.C19 fusion protein. The left panel (+) is serum produced following immunization with an NSP2-NSP1.C19 fusion with adjuvant. The right panel (−) is serum produced following immunization without adjuvant. The center panel is a rabbit polyclonal antibody raised against the SA11 C19 peptide. Pre- and post-immunization serum lanes are indicated. Mock infected cell lysates (negative control) are shown in lanes “1.” SA11 virus infected cell lysates are shown in lanes “2.” Lanes containing molecular weight standards are indicated by an “M.”

FIG. 7 is a bar graph showing that a high titer of serum antibodies is generated against the VP8 component of a VP8-NSP2 platform complex when administered to guinea pigs with and without adjuvant (adj). In the sandwich ELISA assay, a broadly cross-reactive anti-human rotavirus antisera was adsorbed to the ELISA plate, followed sequentially by immunoadsorption of (1) RRV, (2) guinea pig serum raised against the NSP2-VP8 fusion protein, and 3) goat anti-guinea pig antibody conjugated to horseradish peroxidase. The color reaction, developed by adding TMB substrate (KPL), was read at OD₄₅₀ₙₘ with an ELISA reader.

### SUMMARY OF THE SEQUENCE LISTING

SEQ ID NO:1 is a polynucleotide sequence encoding a rotavirus NSP2 polypeptide (SA11).

SEQ ID NO:2 is the amino acid sequence of rotavirus strain SA11 NSP2 polypeptide.

SEQ ID NO:3 is the polynucleotide sequence encoding the Bristol strain NSP2 polypeptide.

SEQ ID NO:4 is the amino acid sequence of the Bristol strain NSP2 polypeptide.

SEQ ID NO:5 is a polynucleotide sequence encoding the C19 peptide of rotavirus (RRV) NSP1.

SEQ ID NO:6 is the amino acid sequence of the RRV NSP1 C19 peptide.

SEQ ID NO:7 is a polynucleotide sequence encoding the C19 peptide of rotavirus (SA11) NSP1.

SEQ ID NO:8 is the amino acid sequence of the SA11 NSP1 C19 peptide.

SEQ ID NO:9 is a polynucleotide sequence encoding the rotavirus strain RRV VP8 polypeptide (VP4 fragment).

SEQ ID NO:10 is the amino acid sequence of the RRV VP8 polypeptide (VP4 fragment).

SEQ ID NO:11 is a polynucleotide sequence encoding a six-histidine tag.

SEQ ID NO:12 is the amino acid sequence of six-histidine tag.

SEQ ID NO:13 is a polynucleotide sequence encoding a GPGP linker peptide.

SEQ ID NO:14 is the amino acid sequence of GPGP linker peptide.

SEQ ID NO:15 is a polynucleotide sequence encoding a NSP2-RRV.NSP1.C19 fusion polypeptide.

SEQ ID NO:16 is the amino acid sequence of a NSP2-RRV.NSP1.C19 fusion polypeptide.

SEQ ID NO:17 is a polynucleotide sequence encoding a NSP2-SA11.NSP1.C19 fusion polypeptide.

SEQ ID NO:18 is the amino acid sequence of a NSP2-SA11.NSP1.C19 fusion polypeptide.

SEQ ID NO:19 is a polynucleotide sequence encoding a NSP2-RRV.VP8 fusion polypeptide with a GPGP linking peptide.

SEQ ID NO:20 is the amino acid sequence of a NSP2-RRV.VP8 fusion polypeptide with a GPGP linking peptide.

SEQ ID NO:21 is a polynucleotide sequence encoding a NSP2-RRV.VP8 fusion polypeptide with a GGS linking peptide.

SEQ ID NO:22 is the amino acid sequence of a NSP2-RRV.VP8 fusion polypeptide with a GGS linking peptide.

SEQ ID NO:23 is a polynucleotide sequence encoding a Histidine tagged Bristol strain NSP2 polypeptide.

SEQ ID NO:24 is the amino acid sequence of a poly-histidine tagged Bristol NSP2 polypeptide.

SEQ ID NO:25 is the forward primer for amplification of the g8 gene.

SEQ ID NO:26 is the reverse primer for amplification of the g8 gene.

SEQ ID NO:27 is the forward primer for RRV NSP1 C19 insertion.

SEQ ID NO:28 is the reverse primer for RRV NSP1 C19 insertion.

SEQ ID NO:29 is the forward primer for SA11 NSP1 C19 insertion.

SEQ ID NO:30 is the reverse primer for SA11 NSP1 C19 insertion.

SEQ ID NO:31 is the forward primer for amplification of the Bristol g9 gene

SEQ ID NO:32 is the reverse primer for amplification of the Bristol g9 gene.

SEQ ID NO:33 is the forward primer for introduction of a NotI restriction site (SA11)

SEQ ID NO:34 is the reverse primer for introduction of a NotI restriction site (SA11).

SEQ ID NO:35 is the forward primer for introduction of a NotI restriction site (Bristol).

SEQ ID NO:36 is the reverse primer for introduction of a NotI restriction site (Bristol).

SEQ ID NO:37 is the polynucleotide sequence of the pQE60 vector.

### DETAILED DESCRIPTION

### Summary of Specific Embodiments

A successful platform for antigen delivery is one that provides 1) multivalent display of antigens; 2) can be efficiently produced and recovered; 3) is physically robust; and 4) can increase the antigen-specific immune responsive relative to the response produced by antigens administered alone or with adjuvants. The present disclosure describes an epitope mounting platform useful as an antigen delivery system, based on viral NSP2 fusion proteins, that possesses many or all of these desirable properties and that can increase the efficacy of immune presentation of an extensive range of antigenic polypeptides. These viral NSP2 fusion proteins are useful for making subunit vaccines, for the production of recombinant antigens, and other purposes.

In an embodiment, the antigen delivery platform comprises a monomeric fusion protein including a) a self-aggregating polypeptide component; b) a linear linking peptide; and c) an antigenic polypeptide. The self-aggregating polypeptide component promotes assembly of monomeric fusion protein subunits into a stable multimeric ring structure that enhances the immune response to an attached antigen. In one embodiment, the monomeric fusion protein includes the following elements linked in an N-terminal to C-terminal direction: (a) a viral NSP2 polypeptide; (b) a linear linking peptide; and, (c) an antigenic polypeptide. Upon expression, multiple monomeric fusion protein subunits form a self-aggregating multimeric ring structure. The self-aggregating multimeric ring structure typically includes 4, 8, 12 or 16 monomeric fusion protein subunits. In an embodiment, the multimeric ring structure is made up of 8 monomeric fusion protein subunits.

For example, in an embodiment the self-aggregating polypeptide component is a rotavirus NSP2 polypeptide. Any rotavirus NSP2 polypeptide, or functional fragment or homolog thereof, can be employed in the fusion proteins described herein. Such an NSP2 polypeptide (or fragment of homolog) is characterized by a histidine triad (HIT)-like fold and forms a self-aggregating multimeric (for example, octameric) ring structure upon expression. For example, the rotavirus NSP2 polypeptide can be selected from a group A rotavirus, a group B rotavirus, a group C rotavirus, a group D rotavirus, a group E rotavirus, a group F rotavirus and a group G rotavirus. In certain exemplary embodiments, the NSP2 polypeptide is a polypeptide with the amino acid sequence of SEQ ID NO:2 or a polypeptide with the sequence of SEQ ID NO:4. In an embodiment, the NSP2 polypeptide is encoded by a nucleic acid with the polynucleotide sequence of SEQ ID NO:1, SEQ ID NO:3, or a polynucleotide sequence that differs from SEQ ID NO:1 or SEQ ID NO:3 solely by virtue of the degeneracy of the genetic code, and therefore encodes the same polypeptide.

Optionally, the fusion protein includes at least one affinity tag to facilitate recovery of fusion protein ring structures when expressed in bacterial or eukaryotic cells. In some embodiments the fusion protein includes more than one affinity tag, for example a first affinity tag adjacent to or contiguous with the linear linker peptide, and a second affinity tag at the C-terminus of the antigenic polypeptide. While various affinity tags are contemplated, in an embodiment, the affinity tag is a poly-histidine tag, such as a six-histidine affinity tag. Similarly, the linear linking peptide can be any of a variety of amino acid sequences. In an embodiment, the linear linking peptide is a glycine-proline-glycine-proline sequence. In another embodiment, the linear linking peptide is a glycine-glycine-serine sequence.

In various embodiments, the antigenic polypeptide is that of a pathogenic organism, such as a viral or bacterial agent that produces undesirable symptoms in a subject following exposure. The antigenic polypeptide can be that of a rotavirus or of a virus other than a rotavirus. A non limiting, and far from exhaustive list of such other viruses includes dengue virus, human immunodeficiency virus, influenza virus, metapneumovirus, norovirus, papillomavirus, parvovirus, SARS virus, smallpox virus, picornaviruses, respiratory syncitial virus, parainfluenza virus, measles, hepatitis, measles, varicella zoster, rabies and West Nile virus. Alternatively, the antigenic polypeptide can be that of a bacteria or other pathogenic organism. Exemplary bacterial polypeptides include the CFP10 polypeptide, or a domain of other polypeptides of Mycobacterium tuberculosis, or of a domain of the pilus polypeptide of Vibrio cholera, the CjaA polypeptide of Campylobacter coli, the Sfb 1 polypeptide of Streptococcus pyogenes, and the UreB polypeptide Helicobacter pylori. Exemplary polypeptides of other pathogenic organisms include the circumsporozoite polypeptide of Plasmodium falciparum. Additionally, the antigenic polypeptide can be a tumor-associated antigen.

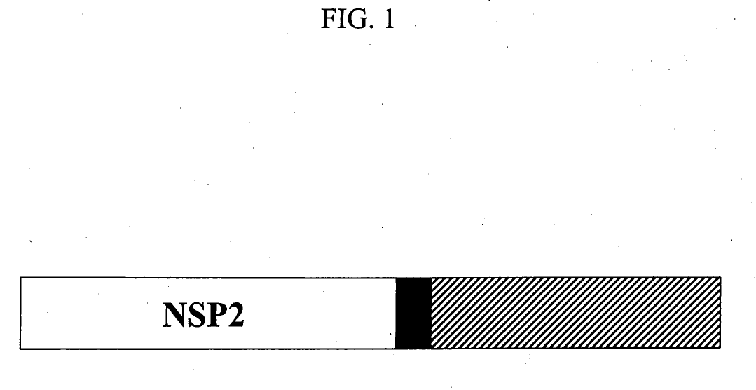

In one exemplary embodiment (shown schematically in FIG. 1), the monomeric fusion protein includes, in an N-terminal to C-terminal direction: a rotavirus NSP2 polypeptide; a linear linking peptide including a six-histidine affinity tag and the amino acid sequence glycine-proline-glycine-proline; and an antigenic polypeptide with a C-terminal six histidine affinity tag. In another exemplary embodiment, the monomeric fusion polypeptide includes a linear linking peptide with the sequence glycine-glycine-serine, and a C-terminal histidine tag. A plurality of such monomeric fusion proteins forms self-aggregating octameric ring structures upon expression in a bacterial or eukaryotic cell, or when expressed following administration to an organism, such as a mammal.

The disclosure also provides isolated or recombinant nucleic acids that include polynucleotides that encode fusion proteins having the following elements linked in an N-terminal to C-terminal direction: (a) a self-aggregating polypeptide component, such as a viral NSP2 polypeptide; (b) a linear linking peptide; and, (c) an antigenic polypeptide. Accordingly, such a nucleic acid includes in a 5′ to 3′ direction: (a) a polynucleotide sequence that encodes a self-aggregating polypeptide, such as a viral NSP2 polypeptide; (b) a polynucleotide sequence that encodes a linear linking peptide; and, (c) a polynucleotide sequence that encodes an antigenic polypeptide.

The polynucleotides encode fusion proteins that form self-aggregating ring structures containing a plurality of monomeric fusion protein subunits. In certain embodiments, the polynucleotides encode fusion proteins that aggregate into ring structures with between 4 and 16 monomeric fusion protein subunits, that is, 4, 8, 12 or 16 subunits, for example, 8 subunits. For example, such polynucleotides can include a polynucleotide that encodes a self-aggregating rotavirus NSP2 polypeptide (or a functional fragment or homolog thereof). Such polynucleotides encode rotavirus NSP2 polypeptides with an HIT-like fold that self-assemble into multimeric ring structures. For example, the polynucleotide can encode a self-aggregating NSP2 polypeptide selected from a group A rotavirus, a group B rotavirus, a group C rotavirus, a group D rotavirus, a group E rotavirus, a group F rotavirus, and a group G rotavirus. In one embodiment, the polynucleotide encodes the NSP2 polypeptide of SEQ ID NO:2 or SEQ ID NO:4, or variants thereof with the same structural and functional properties. For example, the polynucleotide can be the polynucleotide of SEQ ID NO:1 or the polynucleotide sequence of SEQ ID NO:3, or a variant thereof that differs only by virtue of the degeneracy of the genetic code.

The portion of the nucleic acid that encodes a linear linking peptide can encode any of a variety of amino acid sequences, so long as the resulting peptide does not interfere with the expression, stability or conformation of the NSP2 polypeptide or the antigenic polypeptide. Exemplary polynucleotides encode the amino acid sequences glycine-proline-glycine-proline and/or glycine-glycine-serine. In some embodiments, the polynucleotides also encode at least one affinity tag. For example, one favorable affinity tag includes a six-histidine tag. The polynucleotide encoding such an affinity tag can be situated at an internal position (for example adjacent to the linking peptide) or can be situated at the 3′ end of the coding sequence.

The polynucleotides encoding monomeric fusion proteins also encode an antigenic polypeptide component contiguous with the aforementioned linear linking peptide. The polynucleotide encoding the antigenic polypeptide can be derived from essentially any source, and frequently is derived from (for example, isolated or purified from, amplified from, or artificially synthesized corresponding to the sequence of) a pathogenic virus or organism, such as a bacteria or parasite. The encoded polypeptide is typically a polypeptide other than NSP2. Alternatively, the polynucleotide can encode a tumor antigen. For example, the polynucleotide can encode an antigenic polypeptide of a rotavirus or of a virus other than a rotavirus, such as dengue virus, human immunodeficiency virus, influenza virus, metapneumovirus, norovirus, papillomavirus, parvovirus, SARS virus, smallpox virus, picornaviruses, respiratory syncitial virus, parainfluenza virus, measles, hepatitis, measles, varicella zoster, rabies and West Nile virus, among many others. Alternatively, the polynucleotide can encode an antigenic polypeptide derived from a bacterium, such as the CFP10 polypeptide, or a domain of other polypeptides of Mycobacterium tuberculosis, or of a domain of the pilus polypeptide of Vibrio cholera, the CjaA polypeptide of Campylobacter coli, the Sfb 1 polypeptide of Streptococcus pyogenes, and the UreB polypeptide Helicobacter pylori. The polynucleotides can also encode antigenic polypeptides of other pathogenic organisms include the circumsporozoite polypeptide of Plasmodium falciparum.

Vectors including such polynucleotides are also described herein.

Immunogenic compositions including multimeric fusion protein ring structures, and immunogenic compositions including polynucleotides encoding such fusion proteins, as described above are also disclosed. Such immunogenic compositions are useful, for example, as vaccines. Similarly, pharmaceutical compositions including the fusion proteins and polynucleotides encoding the fusion proteins are described herein. Optionally, the pharmaceutical compositions include a carrier or excipient, and can also include additional components, such as aluminum hydroxylphosphosulfate, alum, diphtheria CRM₁₉₇, liposomes, and the like.

Methods of using the fusion proteins and polynucleotides described herein to generate an immune response are also provided. Such methods involve administering a polynucleotide encoding a monomeric NSP2 fusion protein, into which an antigenic polypeptide of interest is incorporated, to an animal, such as a human subject or patient. Alternatively, the methods involve administering fusion protein ring structures recovered following expression of polynucleotides encoding such NSP2 fusion proteins. The fusion protein serves as an antigen delivery platform to enhance the immune response generated against the antigenic polypeptide component of the fusion protein.

### II. Terms

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In order to facilitate review of the various embodiments of this disclosure, the following explanations of specific terms are provided:

Adjuvant: A vehicle used to enhance antigenicity; such as a suspension of minerals (alum, aluminum hydroxide, or phosphate) on which antigen is adsorbed; or water-in-oil emulsion in which antigen solution is emulsified in mineral oil (Freund incomplete adjuvant, montanide-ISA), sometimes with the inclusion of killed mycobacteria (Freund's complete adjuvant) to further enhance antigenicity (inhibits degradation of antigen and/or causes influx of macrophages). In one example, the adjuvant is a mixture of detergents and lipids, such as PROVAX®.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. An “antigenic polypeptide” is a polypeptide to which an immune response, such as a T cell response or an antibody response, can be stimulated. “Epitope” or “antigenic determinant” refers to a site on an antigen to which B and/or T cells respond. In one embodiment, T cells respond to the epitope when the epitope is presented in conjunction with an MHC molecule. Epitopes can be formed both from contiguous amino acids or noncontiguous amino acids juxtaposed by tertiary folding of an antigenic polypeptide. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and multi-dimensional nuclear magnetic resonance spectroscopy.

An antigenic polypeptide can include a virus-specific antigen, an organism-specific antigen or a disease-specific antigen. These terms are not mutually exclusive, as a virus-specific antigen can also be a disease-specific antigen. A virus-specific antigen is an antigenic epitope encoded by the viral genome. A disease-specific antigen is expressed coincidentally with a disease process. A disease-specific antigen may be an antigen recognized by T cells or B cells. For purposes of this disclosure, an organism-specific antigen includes an antigen of a unicellular or multicellular organism, such as a bacteria or a eukaryotic cell or organism. An organism-specific antigen also includes a virus-specific antigen unless otherwise provided.

Antigen Delivery Platform or Epitope Mounting Platform: In the context of the present disclosure, the terms antigen delivery platform and epitope mounting platform refer to a macromolecular complex including one or more antigenic epitopes. Delivery of an antigen (including one or more epitopes) in the context of an epitope mounting platform enhances, increases, ameliorates or otherwise improves a desired antigen-specific immune response to the antigenic epitope(s). The molecular constituents of the antigen delivery platform may be antigenically neutral or may be immunologically active, that is, capable of generating a specific immune response. Nonetheless, the term antigen delivery platform is utilized to indicate that a desired immune response is generated against a selected antigen that is a component of the macromolecular complex other than the platform polypeptide to which the antigen is attached. Accordingly, the epitope mounting platform is useful for delivering a wide variety of antigenic epitopes, including antigenic epitopes of pathogenic organisms such as bacteria and viruses. The antigen delivery platform of the present disclosure is particularly useful for the delivery of complex peptide or polypeptide antigens, which may include one or many distinct epitopes.

Amplification: Of a nucleic acid molecule (e.g., a DNA or RNA molecule) refers to use of a technique that increases the number of copies of a nucleic acid molecule in a specimen. An example of amplification is the polymerase chain reaction (PCR), in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification may be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Antibody: Immunoglobulin molecules and immunologically active portions of immunoglobulin molecules, that is, molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the V_{L}, V_{H}, C_{L} and C_{H1} domains; (ii) an F_{d} fragment consisting of the V_{H} and C_{H1} domains; (iii) an Fv fragment consisting of the V_{L} and V_{H} domains of a single arm of an antibody, (iv) a dAb fragment (Ward et al., Nature 341:544-546, 1989) which consists of a V_{H} domain; (v) an isolated complimentarity determining region (CDR); and (vi) a F(ab′)₂ fragment, a bivalent fragment comprising two Fab fragments linked by a disulfide bridge at the hinge region.

Methods of producing polyclonal and monoclonal antibodies are known to those of ordinary skill in the art, and many antibodies are available. See, e.g., Coligan, Current Protocols in Immunology Wiley/Greene, NY, 1991; and Harlow and Lane, Antibodies: A Laboratory Manual Cold Spring Harbor Press, NY, 1989; Stites et al., (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y.1986; and Kohler and Milstein, Nature 256: 495-497, 1975. Other suitable techniques for antibody preparation include selection of libraries of recombinant antibodies in phage or similar vectors. See, Huse et al., Science 246: 1275-1281, 1989; and Ward et al., Nature 341: 544-546, 1989. “Specific” monoclonal and polyclonal antibodies and antisera (or antiserum) will usually bind with a K_{D} of at least about 0.1 μM, preferably at least about 0.01 μM or better, and most typically and preferably, 0.001 μM or better.

Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984). Detailed methods for preparation of chimeric (humanized) antibodies can be found in U.S. Pat. No. 5,482,856. Additional details on humanization and other antibody production and engineering techniques can be found in Borrebaeck (ed), Antibody Engineering, 2^{nd} Edition Freeman and Company, NY, 1995; McCafferty et al., Antibody Engineering, A Practical Approach, IRL at Oxford Press, Oxford, England, 1996, and Paul Antibody Engineering Protocols Humana Press, Towata, N.J., 1995.

Animal: Living multi-cellular vertebrate organisms, a category that includes, for example, mammals and birds. The term mammal includes both human and non-human mammals. Similarly, the term “subject” includes both human and veterinary subjects.

Conservative variants: “Conservative” amino acid substitutions are those substitutions that do not substantially affect or decrease a desired activity of a protein or polypeptide. For example, in the context of the present disclosure, a conservative amino acid substitution does not substantially alter or decrease the immunogenicity of an antigenic epitope. Similarly, a conservative amino acid substitution does not substantially affect the structure or, for example, the stability of a protein or polypeptide. Specific, non-limiting examples of a conservative substitution include the following examples:

| Original Residue | Conservative Substitutions |
|---|---|
| Ala | Ser |
| Arg | Lys |
| Asn | Gln; His |
| Asp | Glu |
| Cys | Ser |
| Gln | Asn |
| Glu | Asp |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that antibodies raised to the substituted polypeptide also immunoreact with the unsubstituted polypeptide. Non-conservative substitutions are those that reduce an activity or antigenicity or substantially alter a structure, such as a secondary or tertiary structure, of a protein or polypeptide.

cDNA (complementary DNA): A piece of DNA lacking internal, non-coding segments (introns) and regulatory sequences that determine transcription. cDNA is typically synthesized in the laboratory by reverse transcription from messenger RNA extracted from cells.

Dendritic cell (DC): Dendritic cells are the principle antigen presenting cells (APCs) involved in primary immune responses. Dendritic cells include plasmacytoid dendritic cells and myeloid dendritic cells. Their major function is to obtain antigen in tissues, migrate to lymphoid organs and present the antigen in order to activate T cells. Immature dendritic cells originate in the bone marrow and reside in the periphery as immature cells.

Diagnostic: Identifying the presence or nature of a pathologic condition, such as, but not limited to a condition induced by a viral or other pathogen. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (or for example, the probability of severity) of a pathologic condition, such as a symptom induced by a viral infection or other pathogenic organism, or resulting indirectly from such an infection.

Epitope: An antigenic determinant. These are particular chemical groups or peptide sequences on a molecule that are antigenic, that is, that elicit a specific immune response. An antibody specifically binds a particular antigenic epitope on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or 8-10 amino acids in a unique spatial conformation. Methods of determining spatial conformation of epitopes include, for example, x-ray crystallography and multi-dimensional nuclear magnetic resonance spectroscopy. See, e.g., “Epitope Mapping Protocols” in Methods in Molecular Biology, Vol. 66, Glenn E. Morris, Ed (1996). In one embodiment, an epitope binds an MHC molecule, e.g., an HLA molecule or a DR molecule. These molecules bind polypeptides having the correct anchor amino acids separated by about eight or nine amino acids

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (typically, ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences.

Promoter: A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (for example, metallothionein promoter) or from mammalian viruses (for example, the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Host cells: Cells in which a polynucleotide, for example, a polynucleotide vector or a viral vector, can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Immune response: A response of a cell of the immune system, such as a B cell, T cell, or monocyte, to a stimulus. In some cases, the response is specific for a particular antigen (that is, an “antigen-specific response”). In some cases, an immune response is a T cell response, such as a CD4+ response or a CD8+ response. Alternatively, the response is a B cell response, and results in the production of specific antibodies. A “protective immune response” is an immune response that inhibits a detrimental function or activity (such as a detrimental effect of a pathogenic organism such as a virus), reduces infection by a pathogenic organism (such as, a virus), or decreases symptoms that result from infection by the pathogenic organism. A protective immune response can be measured, for example, by the inhibition of viral replication or plaque formation in a plaque reduction assay or ELISA-neutralization assay (NELISA), or by measuring resistance to viral challenge in vivo.

Immunogenic peptide: A peptide which comprises an allele-specific motif or other sequence such that the peptide will bind an MHC molecule and induce a cytotoxic T lymphocyte (“CTL”) response, or a B cell response (e.g. antibody production) against the antigen from which the immunogenic peptide is derived.

Immunogenic composition: A composition comprising at least one epitope of a virus, or other pathogenic organism, that induces a measurable CTL response, or induces a measurable B cell response (for example, production of antibodies that specifically bind the epitope). It further refers to isolated nucleic acids encoding an immunogenic epitope of virus or other pathogen that can be used to express the epitope (and thus be used to elicit an immune response against this polypeptide or a related polypeptide expressed by the pathogen). For in vitro use, the immunogenic composition may consist of the isolated nucleic acid, protein or peptide. For in vivo use, the immunogenic composition will typically include the nucleic acid, protein or peptide in pharmaceutically acceptable carriers or excipients, and/or other agents, for example, adjuvants. An immunogenic polypeptide (such as an antigenic polyeptide), or nucleic acid encoding the polypeptide, can be readily tested for its ability to induce a CTL or antibody response by art-recognized assays.

Isolated: An “isolated” biological component (such as a nucleic acid or protein or organelle) has been substantially separated or purified away from other biological components in the cell of the organism in which the component naturally occurs, for example, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule to facilitate detection of that molecule. Specific, non-limiting examples of labels include fluorescent tags, affinity tags, enzymatic linkages, and radioactive isotopes. An affinity tag is a peptide or polypeptide sequence capable of specifically binding to a specified substrate, for example, an organic, non-organic or enzymatic substrate or cofactor. A polypeptide including a peptide or polypeptide affinity tag can typically be recovered, for example, purified or isolated, by means of the specific interaction between the affinity tag and its substrate. An exemplary affinity tag is a poly-histidine (e.g., six-histidine) affinity tag which can specifically bind to non-organic metals such as nickel and/or cobalt. Additional affinity tags are well known in the art.

Linking peptide: A linking peptide (or linker sequence) is an amino acid sequence that covalently links two polypeptide domains. Linking peptides can be included between the rotavirus NSP2 polypeptide and an antigenic epitope to provide rotational freedom to the linked polypeptide domains and thereby to promote proper domain folding. Linking peptides, which are generally between 2 and 25 amino acids in length, are well known in the art and include, but are not limited to the amino acid sequences glycine-proline-glycine-proline (GPGP) and glycine-glycine-serine (GGS), as well as the glycine(4)-serine spacer described by Chaudhary et al., Nature 339:394-397,1989. In some cases multiple repeats of a linking peptide are present.

Lymphocytes: A type of white blood cell that is involved in the immune defenses of the body. There are two main types of lymphocytes: B cells and T cells.

Mammal: This term includes both human and non-human mammals unless otherwise specified. Similarly, the term “subject” includes both human and veterinary subjects.

Oligonucleotide: A linear polynucleotide sequence of up to about 100 nucleotide bases in length.

Open reading frame (“ORF”): A series of nucleotide triplets (codons) coding for amino acids without any internal termination codons. These sequences are usually translatable into a polypeptide (peptide or protein).

Operatively linked: A first nucleic acid sequence is operatively linked with a second nucleic acid sequence when the first nucleic acid sequence is placed in a functional relationship with the second nucleic acid sequence. For instance, a promoter is operatively linked to a coding sequence if the promoter affects the transcription or expression of the coding sequence. Generally, operatively linked DNA sequences are contiguous and, where necessary to join two protein-coding regions, in the same reading frame, for example, two polypeptide domains or components of a fusion protein.

Pharmaceutically acceptable carriers and/or pharmaceutically acceptable excipients: The pharmaceutically acceptable carriers or excipients of use are conventional. Remington's Pharmaceutical Sciences, by E. W. Martin, Mack Publishing Co., Easton, Pa., 15th Edition (1975), describes compositions and formulations suitable for pharmaceutical delivery of the polypeptides and polynucleotides disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition used to achieve a desired effect in a subject. For instance, this can be the amount of the composition necessary to inhibit viral (or other pathogen) replication or to prevent or measurably alter outward symptoms of viral (or other pathogenic) infection. When administered to a subject, a dosage will generally be used that will achieve target tissue concentrations (for example, in lymphocytes) that has been shown to achieve an in vitro effect.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Polypeptide: Any chain of amino acids, regardless of length or post-translational modification (for example, glycosylation or phosphorylation), such as a protein or a fragment or subsequence of a protein. The term “peptide” is typically used to refer to a chain of amino acids of between 3 and 30 amino acids in length. For example an immunologically relevant peptide may be between about 7 and about 25 amino acids in length, e.g., between about 8 and about 10 amino acids.

In the context of the present disclosure, a polypeptide can be a fusion protein comprising a plurality of constituent polypeptide (or peptide) elements. Typically, the constituents of the fusion protein are genetically distinct, that is, they originate from distinct genetic elements, such as genetic elements of different organisms or from different genetic elements (genomic components) or from different locations on a single genetic element, or in a different relationship than found in their natural environment. Nonetheless, in the context of a fusion protein the distinct elements are translated as a single polypeptide. The term monomeric fusion protein (or monomeric fusion protein subunit) is used synonymously with such a single fusion protein polypeptide to clarify reference to a single constituent subunit where the translated fusion proteins assume a multimeric tertiary structure.

Specifically, in an embodiment, a monomeric fusion protein subunit includes in an N-terminal to C-terminal direction: a viral NSP2 polypeptide; a linear linking peptide; and an antigenic polypeptide or epitope translated into a single polypeptide monomer. A plurality (for example, 4, 8, 12 or 16) of monomeric fusion protein subunits self-assembles into a multimeric ring structure.

Preventing or treating a disease: Inhibiting infection by a pathogen such as a virus, such as a rotavirus or other virus, refers to inhibiting the full development of a disease. For example, inhibiting a viral infection refers to lessening symptoms resulting from infection by the virus, such as preventing the development of symptoms in a person who is known to have been exposed to the virus, or to lessening virus number or infectivity of a virus in a subject exposed to the virus. “Treatment” refers to a therapeutic or prophylactic intervention that ameliorates or prevents a sign or symptom of a disease or pathological condition related to infection of a subject with a virus or other pathogen.

Probes and primers: A probe comprises an isolated nucleic acid attached to a detectable label or reporter molecule. Primers are short nucleic acids, preferably DNA oligonucleotides, for example, a nucleotide sequence of about 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example, by the polymerase chain reaction (PCR) or other nucleic-acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only about 15 nucleotides. Thus, in order to obtain greater specificity, probes and primers may be selected that comprise 20, 25, 30, 35, 40, 50 or more consecutive nucleotides.

Promoter: A promoter is an array of nucleic acid control sequences that directs transcription of a nucleic acid. A promoter includes necessary nucleic acid sequences near the start site of transcription, such as in the case of a polymerase II type promoter (a TATA element). A promoter also optionally includes distal enhancer or repressor elements which can be located as much as several thousand base pairs from the start site of transcription. Both constitutive and inducible promoters are included (see e.g., Bitter et al., Methods in Enzymology 153:516-544, 1987).

Specific, non-limiting examples of promoters include promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Protein purification: the fusion polypeptides disclosed herein can be purified (and/or synthesized) by any of the means known in the art (see, e.g., Guide to Protein Purification, ed. Deutscher, Meth. Enzymol. 185, Academic Press, San Diego (1990); and Scopes, Protein Purification: Principles and Practice, Springer Verlag, New York (1982). Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Purified: The term “purified” does not require absolute purity; rather, it is intended as a relative term. Thus, for example, a purified nucleic acid is one in which the nucleic acid is more enriched than the nucleic acid in its natural environment within a cell. Similarly, a purified peptide preparation is one in which the peptide or protein is more enriched than the peptide or protein is in its natural environment within a cell. In one embodiment, a preparation is purified such that the protein or peptide represents at least 50% (such as, but not limited to, 70%, 80%, 90%, 95%, 98% or 99%) of the total peptide or protein content of the preparation.

Recombinant: A recombinant nucleic acid is one that has a sequence that is not naturally occurring or has a sequence that is made by an artificial combination of two otherwise separated segments of sequence, for example, a polynucleotide encoding a fusion protein. This artificial combination is often accomplished by chemical synthesis or, more commonly, by the artificial manipulation of isolated segments of nucleic acids, e.g., by genetic engineering techniques.

Sequence identity: The similarity between amino acid (and polynucleotide) sequences is expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two amino acid sequences, the more similar are the higher order structures resulting from folding and assembly. However, the converse is not necessarily true, and polypeptides with low sequence identity at the amino acid level can nonetheless have highly similar tertiary and quaternary structures. For example, NSP2 homologs with little sequence identity (for example, less than 50% sequence identity, or even less than 30%, or less than 20% sequence identity) share similar higher order structure and assembly properties, such that even distantly related NSP2 proteins assemble into multimeric ring structures as described herein.

Methods of determining sequence identity are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Another indicia of sequence similarity between two nucleic acids is the ability to hybridize. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning. A Laboratory Manual, Cold Spring Harbor Laboratory Press, Cold Spring Harbor, N.Y., 2001; Tijssen, Hybridization With Nucleic Acid Probes, Part I: Theory and Nucleic Acid Preparation, Laboratory Techniques in Biochemistry and Molecular Biology, Elsevier Science Ltd., NY, N.Y., 1993.and Ausubel et al. Short Protocols in Molecular Biology, 4^{th} ed., John Wiley & Sons, Inc., 1999.

For purposes of the present disclosure, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast nucleic acids that hybridize under “low stringency conditions include those with much less sequence identity, or with sequence identity over only short subsequences of the nucleic acid.

For example, a specific example of progressively higher stringency conditions is as follows: 2× SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N.Y., 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Subject: Living multi-cellular vertebrate organisms, a category that includes both human and veterinary subjects, including human and non-human mammals.

T Cell: A white blood cell critical to the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8 T cell is a cytotoxic T lymphocyte. In another embodiment, a CD8 cell is a suppressor T cell.

Therapeutically active polypeptide: An agent, such as an epitope of a virus or other pathogen that causes induction of an immune response, as measured by clinical response (for example increase in a population of immune cells, increased cytolytic activity against the epitope). Therapeutically active molecules can also be made from nucleic acids. Examples of a nucleic acid based therapeutically active molecule is a nucleic acid sequence that encodes an epitope of a protein of a virus or other pathogen, wherein the nucleic acid sequence is operatively linked to a control element such as a promoter.

In one embodiment, a therapeutically effective amount of an antigenic epitope is an amount used to generate an immune response, or inhibit a function or activity of a virus or other pathogen. Treatment refers to a therapeutic intervention that ameliorates a sign or symptom resulting from exposure to a virus or other pathogen, or a reduction in viral or pathogen load. Treatment also refers to a prophylactic intervention to prevent a sign or symptom that results from exposure to a virus or other pathogen, or to reduce viral or pathogen load.

Transduced or Transfected: A transduced cell is a cell into which a nucleic acid molecule has been introduced by molecular biology techniques. As used herein, the term introduction or transduction encompasses all techniques by which a nucleic acid molecule might be introduced into such a cell, including transfection with viral vectors, transformation with plasmid vectors, and introduction of naked DNA by electroporation, lipofection, and particle gun acceleration.

Vaccine: A vaccine is a pharmaceutical composition that elicits a prophylactic or therapeutic immune response in a subject. In some cases, the immune response is a protective immune response. Typically, a vaccine elicits an antigen-specific immune response to an antigen of a pathogen, for example, a bacterial or viral pathogen, or to a cellular constituent correlated with a pathological condition. A vaccine may include a polynucleotide, a peptide or polypeptide, a virus, a bacteria, a cell or one or more cellular constituents. In some cases, the virus, bacteria or cell may be inactivated or attenuated to prevent or reduce the likelihood of infection, while maintaining the immunogenicity of the vaccine constituent.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker gene and other genetic elements known in the art.

### III. Detailed Description of Several Embodiments

### Characteristics of the Rotavirus NSP2 Protein

The NSP2 protein has a mass of 35 kDa, and is a nonspecific ssRNA-binding protein with two distinct domains, an N-terminal domain from amino acid residues 1 to 141, and a C-terminal domain from amino acids 151-317 that includes a histidine triad (HIT)-like motif. The NSP2 protein self-assembles into stable “doughnut” or ring-shaped multimers that possess NTPase activity.

In the fusion proteins described herein, the unstructured surface of the exposed C-terminus of the NSP2 serves as a site for the insertion of an antigenic epitope, such as a peptide or polypeptide derived from a pathogenic organism, for example, a pathogenic virus, to which an immune response is desirable.

Due to extensive inter-subunit interactions, the NSP2 multimer is highly stable under a broad range of pH (5-9), temperatures, and ionic conditions (50-250 mM NaCl), facilitating large scale protein production and purification, and enabling storage under conditions suitable for pharmaceutical compositions, for example, vaccine compositions.

### Fusion Polypeptides

Embodiments described in the present disclosure are fusion proteins useful as antigen delivery platforms for the production of vaccine compositions to elicit an immune response in a subject, such as a human subject. Accordingly, certain embodiments, including polynucleotides encoding fusion proteins as well as the expressed fusion proteins are suitable as vaccine compositions, and can be used to elicit a protective immune response, for example, against a virus or other pathogen from which the antigen was derived. The immune response may be desirable to prevent or reduce the symptoms typically resulting from exposure to an infectious agent, for example, infection with a pathogen, such as a bacterial or viral pathogen. In other instances, the immune response can be desirable to reduce the presence of or prevent the occurrence of a tumor. Accordingly, the compositions described herein, including an antigen delivery platform can be administered prophylactically prior to the exposure to the infectious agent. Alternatively, the immune response may be desirable to ameliorate symptoms resulting from exposure. In this case compositions including the antigen delivery platforms described herein can be administered subsequent to exposure, for example, therapeutically administered. Compositions, including vaccine compositions incorporating the antigen delivery platforms, that is, fusion proteins and/or polynucleotides encoding fusion proteins are detailed herein below.

The fusion proteins described herein include a) a self-aggregating polypeptide component; b) a linear linking peptide; and, c) an antigenic polypeptide component, arranged in an N-terminal to C-terminal orientation. In the context of the present disclosure, the self-aggregating polypeptide component is a viral NSP2 polypeptide, such as a rotavirus NSP2 polypeptide, or a functional fragment, variant, homolog or analog thereof. The NSP2 fusion polypeptides described herein assemble into stable multimeric ring structures. Typically, the structure is an octamer formed by the interaction of two NSP2 tetramers arranged in a tail-to-tail orientation. However, alternative aggregates of tetramers and/or octamers are also suitable.

Provided that the multimeric, (typically, octameric) ring structure is retained, any functional fragment, homolog or variant of an NSP2 polypeptide can be employed in the fusion proteins and methods described herein. NSP2 polypeptides suitable for use in the fusion polypeptides and immunogenic compositions described herein can diverge substantially at the amino acid sequence level, so long as specific structural and functional characteristics are maintained. Specifically, the NSP2 polypeptides of the present disclosure include a characteristic HIT-like fold. The HIT-like motif is a three-dimensional structural motif discernable at the level of tertiary structure. The classic cellular histidine triad (HIT) motif is a conserved protein structural motif that has been identified in a family of nucleotide interacting proteins (see, e.g., Lima et al., Science 278:286-290, 1997; Lima et al., Proc. Natl. Acad. Sci USA 28:5357-5362, 1996). NSP2 polypeptides do not share significant amino acid identity or similarity with proteins having the HIT motif, however, superimposition of their tertiary (folded) structures reveals a high degree of structural similarity. It is this HIT-like structural motif or fold that is conserved between NSP2 proteins favorably utilized in the fusion polypeptides described herein. In wild type NSP2 proteins, the HIT-like motif confers NTPase activity characterized by hydrolysis of the γ-phosphate of nucleoside triphosphates. This activity is not essential for proper folding of the NSP2 octamer. Thus, NSP2 polypeptides that possess a properly folded HIT-motif, regardless of whether they possess NTPase activity, are suitable in the context of the fusion polypeptides described herein. For example, in addition to full length NSP2 polypeptides, fragments of NSP2 polypeptides that include a HIT-like fold and self assemble into stable multimeric ring structures can be used in the fusion proteins and immunogenic and other compositions described herein. For example, the C-terminus of the NSP2 polypeptide is relatively amenable to modification, and fragments of NSP2 proteins lacking one or more C-terminal amino acids are suitable for use in the fusion proteins disclosed herein.

To date, at least seven distinguishable classes of rotavirus have been identified (designated A-G), and it is likely that additional classes will be defined. Comparison of the homologs and variants of the NSP2 polypeptide encoded by the group A rotaviruses demonstrates an amino acid sequence identity and sequence similarity of as low as 58% and 74%, respectively, depending on the strains compared. Comparison of the NSP2 polypeptides of the group A and group C rotaviruses shows an amino acid sequence identity and sequence similarity of as low as 34% and 55%, respectively. Finally, comparison of the NSP2 polypeptides of the group A and group B rotaviruses show they have an amino acid sequence identity and sequence similarity of as low as 17% and 32%, respectively. Despite the limited degree of sequence conservation for the NSP2 polypeptide among the different groups of rotaviruses (including A, B, C, D, E, F, G), the ability of the NSP2 polypeptide to form the octameric aggregate is retained. Thus, NSP2 polypeptides derived from any of these (or indeed from yet to be described NSP2 polypeptides) that share the above-described properties are suitable in the context of the antigen delivery platforms described herein.

Accordingly, the NSP2 polypeptide can be selected from any of numerous strains of rotavirus known in the art are suitable for use in the fusion proteins described herein. Non-limiting examples of favorable NSP2 polypeptides include those of Group A simian rotavirus (for example, SA11 strain) and reassortant viruses, such as the D×RRV reassortant strain, as well as those of Group C rotaviruses, such as that of the Bristol strain. Alternatively, the NSP2 protein can be selected from a murine (e.g., McNeal et al., Virology 320:1-11, 2004), porcine (GENBANK® Accession No. X06722), bovine (GENBANK® Accession No. Z21640; U.S. Pat. No. 5,626,857), avian (GENBANK® Accession No. AB009625), or human (A, B, C, D, E, F and/or G strain, including, for example, GENBANK® Accession Nos. AF506018; AF506293; AY238393; AY238383; AY212934; AJ132205; AF205850; AB022770; AY539861 and X94562) rotavirus. Additional functional homologues, such as the NS2 protein of bluetongue virus (e.g., GENBANK® Accession Nos. AY124372; X13374; D00500; AY138896 and X58064) can also be used in the fusion proteins described herein. Each of the preceding sequences represented herein by Accession Number are effective as of the filing date of U.S. Provisional Application 60/633,036 (Dec. 3, 2004). The sequences represented by the preceding Accession Numbers and U.S. Provisional Application 60/633, 036 are incorporated herein by reference in their entirety for all purposes.

Because many human subjects are exposed to Group A rotaviruses early in childhood, and have circulating antibodies that may interfere with the efficacy of an antigen delivery platform based on a Group rotavirus NSP2 polypeptide, it can be desirable to substitute an NSP2 polypeptide that is not bound by antibodies raised in response to Group A rotavirus NSP2. For example, Group C rotavirus only rarely infects or causes disease in humans, and most human subjects have no circulating antibodies that bind to NSP2 protein of this rotaviruses group. Thus, in applications where it is desired to avoid immunosurveillance in humans, the self-aggregating polypeptide component can be advantageously selected from a Group C rotavirus, such as the Bristol strain NSP2 polypeptide represented by SEQ ID NO:4. Despite substantial amino acid differences, the Group C NSP2 protein forms multimers that are highly similar to those formed by Group A NSP2, and which are suitable as the self-aggregating polypeptide component of an antigen delivery platform.

In some embodiments, the fusion protein includes at least one amino acid sequence suitable as an affinity tag or label. Such an affinity tag facilitates purification or isolation of the fusion protein. Numerous amino acid sequences ranging from short peptides, such as poly-histidine, e.g., six- histidine amino acid sequences, to longer polypeptides with enzymatic activity, such as the bacterial glutathione-S-transferase (GST) polypeptide, are known in the art. In the context of the present disclosure, any affinity tag that does not interfere with folding or stability of the fusion protein can be employed. Exemplary embodiments described in greater detail herein typically utilize a poly-histidine, e.g., six-histidine, affinity tag. The affinity tag can be situated at an internal position within the fusion polypeptide, such as between the NSP2 polypeptide and the antigenic polypeptide (for example adjacent to the linking peptide) or at the terminus (for example the C-terminus) of the fusion polypeptide. In some embodiments, multiple affinity tags are included. In such cases, the multiple tags can be internal, terminal, or a combination thereof. Recovery of the expressed fusion protein can be accomplished using methods well known in the art such as affinity chromatography using a resin bearing an affinity partner or substrate of the affinity tag. For example, a protein having a poly-histidine affinity tag can be recovered by affinity chromatography (or other binding procedure) using a resin or matrix (e.g., agarose) coupled to a metal moiety such as nickel or cobalt. A polypeptide with a GST component can be recovered via binding to substrate-bound matrix.

Between the self-aggregating polypeptide component and the antigenic polypeptide, the fusion protein includes a linker sequence or linear linking peptide, that is, a short amino acid sequence providing a flexible linker that permits attachment of an antigenic epitope without disruption of the structure, aggregation (multimerization) or activity of the self-aggregating polypeptide component. Typically, a linear linking peptide consists of between two and 25 amino acids. Usually, the linear linking peptide is between three and 15 amino acids in length. For example, the linear linking peptide can be a short sequence of alternating glycines and prolines, such as the amino acid sequence glycine-proline-glycine-proline. A linking peptide can also consist of one or more repeats of the sequence glycine-glycine-serine. Alternatively, the linear linking peptide can be somewhat longer, such as the glycine(4)-serine spacer described by Chaudhary et al., Nature 339:394-397,1989.

Directly or indirectly adjacent to the remaining end of the linear linking peptide (that is, the end of the linear linking peptide not attached to the self-aggregating polypeptide component of the fusion protein) is a polypeptide sequence including at least one antigenic epitope. Such an antigenic polypeptide may be a short peptide sequence including a single epitope. For example the antigenic polypeptide can be a sequence of amino acids as short as eight or nine amino acids, sufficient in length to provide an antigenic epitope in the context of presentation by a cellular antigen presenting complex, such as the major histocompatibility complex (MHC). Larger peptides, in excess of 10 amino acids, 20 amino acids or 30 amino acids are also suitable antigenic polypeptides, as are much larger polypeptides provided that the antigenic polypeptide does not disrupt the structure or aggregation of the NSP2 polypeptide component. Exemplary embodiments ranging from short peptides (for example, less than 20 amino acids in length) to large polypeptides (for example, greater than 150 amino acids), including multiple antigenic epitopes and having a complex secondary structure, are described in the examples herein.

In various embodiments, the antigenic polypeptide is that of a pathogenic organism, such as a virus or bacterial agent that can produce undesirable symptoms in a subject following exposure to the organism. The antigenic polypeptide can be that of a rotavirus, or of a virus other than a rotavirus, such as dengue virus, human immunodeficiency virus, influenza virus, metapneumovirus, norovirus, papillomavirus, parvovirus, SARS virus, smallpox virus, foot and mouth disease virus or West Nile virus. Examples of viral antigens suitable for the production of vaccines in the context of the fusion proteins described herein are described in the following patents and publications: U.S. Pat. Nos. 6,589,529; 6,086,880; and 5,298,244 (rotavirus); U.S. Pat. Nos. 6,514,501 and 6,455,509, and Osatomi, Virology 176:643-647, 1980 (dengue virus); U.S. Pat. No. 6,706,859 (HIV gagp24); U.S. Pat. No. 6,692,955 (HIV gp160, gp120, gp41); U.S. Pat. No. 6,090,392 (HIV gp120); published U.S. patent applications 20040005544 and 20030343436 (metapneumovirus); Mason et al., Proc. Natl. Acad. Sci USA 93:5335-5340, 1996; Tacket et al., J. Inf. Dis. 181 Suppl. 2:S387-391, 2000 (norovirus); U.S. Pat. Nos. 6,551,597 and 6,183,745 (papillomavirus); U.S. Pat. Nos. 6,063,385; 6,458,362; and 6,379,885 (parvovirus); Pand et al., J. Gen. Virol. 85:3109-3113, 2004 (SARS); and, published U.S. patent application No. 20040037848 (West Nile virus). Additionally, influenza hemagluttanin (HA) and neuraminidase (NA) antigens corresponding to relevant strains selected on an annual basis (for example, by the World Health Organization) can be presented in the context of the antigen delivery platform described herein. The sequences of exemplary viral antigens provided in these patents and publications are incorporated by reference herein for all purposes.

In certain examples, the antigenic polypeptide is derived from a rotavirus. Typically, the rotavirus antigen is a peptide or polypeptide selected from a protein other than NSP2. For example, in an embodiment, the antigenic polypeptide is a rotavirus NSP1 polypeptide, for example the NSP1 polypeptides of SEQ ID NO:6 and/or SEQ ID NO:8, or a homolog thereof with at least 90% (or at least 95%, or at least 99%) sequence identity to the polypeptide of SEQ ID NO:6 and or SEQ ID NO:8. In an embodiment, the NSP1 polypeptide is encoded by a polypeptide with at least 75% sequence identity to SEQ ID NO:5 and/or SEQ ID NO:7, for example a polypeptide with at least 80%, or at least 90%, or even 95%, or 99% identity to SEQ ID NO:5 and/or SEQ ID NO:7. In another embodiment, the antigenic polypeptide is a rotavirus VP8 polypeptide or a fragment or subsequence thereof, such as the VP8 polypeptide of SEQ ID NO:10 or a homolog thereof with at least 90% (or at least 95%, or at least 99%) sequence identity to the polyeptide of SEQ ID NO:10. For example, the VP8 polypeptide can be encoded by a polynucleotide with the sequence of SEQ ID NO:9, or a polynucleotide with at least 75%, 80%, 90%, 95% or 99% sequence identity thereto.

Alternatively, the antigenic polypeptide can be that of a bacteria such as the CFP10 polypeptide or a domain of other polypeptides of Mycobacterium tuberculosis, or of a domain of the pilus polypeptide of Vibrio cholera, the CjaA polypeptide of Campylobacter coli, the Sfb1 polypeptide of Streptococcus pyogenes, the UreB polypeptide Helicobacter pylori, or of other pathogenic organisms such as the circumsporozoite polypeptide of Plasmodium falciparum, or can represent a tumor-associated antigens. Non-limiting examples of bacterial (including mycobacterial) epitopes can be found, for example, in Mei et al., Mol. Microbiol. 26:399-407, 1997; and U.S. Pat. No. 6,790,950 (gram negative bacteria); U.S. Pat. No. 6,790,448 (gram positive bacteria); U.S. Pat. Nos. 6,776,993 and 6,384,018 (Mycobacterium tuberculosis). In addition, appropriate tumor-associated antigens or domain thereof, such as carcinoembryonic antigen (“CEA:” e.g., GENBANK® Accession No. AAA62835), ras proteins (see, e.g., Parada et al. Nature 297:474-478, 1982), p53 protein (e.g., GENBANK® Accession No. P07193), prostate-specific antigen (“PSA:” e.g., GENBANK® Accession Nos. NP001639, NP665863), Muc1 (e.g., GENBANK® Accession No. P15941), tyrosinase (see, e.g., Kwon et al., Proc Natl Acad Sci USA 84:7473-7477, 1987, erratum Proc Natl Acad Sci USA 85:6352, 1988 Melanoma-associated antigen (MAGEs: for examples, see, U.S. Pat. Nos: 5,462,871; 5,554,724; 5,554,506; 5,541,104 and 5,558,995), can be mounted on NSP2 platforms. The exemplary polynucleotide and amino acid sequences disclosed in each of these patents and publications, and represented by each of the GENBANK® Accession Numbers as of Dec. 3, 2004, are incorporated herein by reference for all purposes. As indicated above with respect to the exemplary rotavirus antigens, polypeptides with substantial sequence identity (for example, greater than 70%, 80%, 90%, 95%, or 99%) to one or more of these antigens are also suitable antigens.

One of skill in the art will recognize that the exemplary antigens provided above are nonlimiting examples of antigens, and that the fusion polypeptides disclosed herein serve as epitope mounting platforms for a wide variety of antigens. In particular, because of the ease with which the fusion proteins can be produced, the antigen delivery platform described herein is particularly suitable for producing vaccines aimed at generating an immune response against a newly emergent viral pathogen, such as the SARS and West Nile virus, as well as rapidly evolving viruses such as influenza. Additionally, the fusion proteins described herein can be used to produce and deliver any antigenic polypeptide (or peptide) to which an immune response is desired for experimental or therapeutic purposes, not limited to the pathogenic organisms described above. The fusion proteins are also an advantageous antigen delivery platform for the production and presentation of cellular antigens, not limited to antigenic peptides or polypeptides of extracellular parasites, multicellular organisms, cancer specific antigens, and the like.

FIG. 1 schematically illustrates an exemplary embodiment of a fusion protein as described herein.

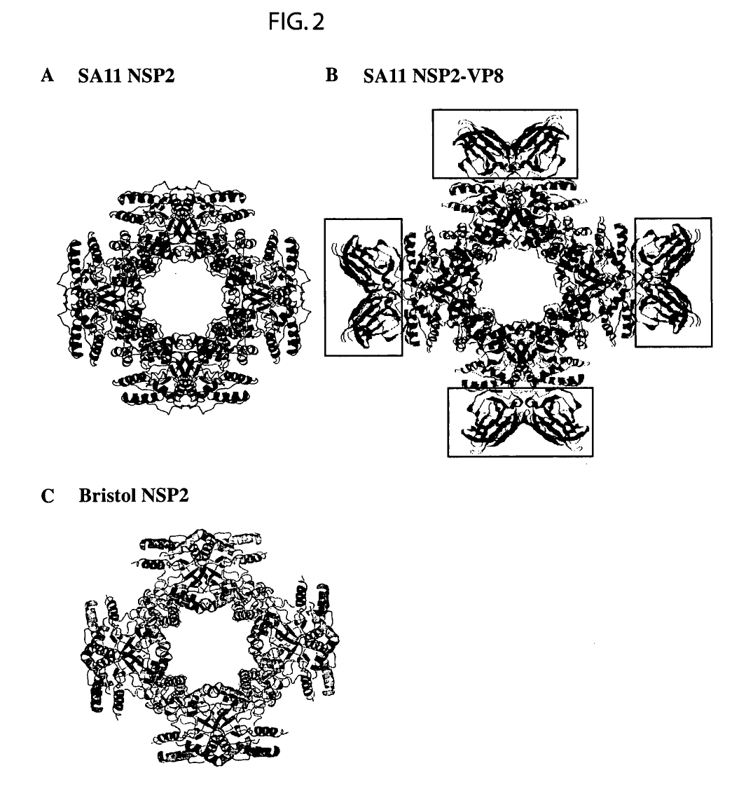

In an embodiment, the fusion protein is a monomeric fusion protein that forms a subunit of a multimeric structure. More particularly, the structure is a ring structure having between four and 16 monomeric fusion protein subunits. Typically, the multimeric structure includes 4, 8, 12 or 16 monomeric fusion protein subunits. For example, in certain embodiments, the multimeric structure is a ring structure having four or eight monomeric fusion protein subunits. In an embodiment, the ring structure is an octamer ring structure consisting of two fusion protein tetramers arranged in a tail-to-tail orientation. As illustrated in FIG. 2, the antigenic polypeptide is exposed on the surface of the ring structure.

Amino Acid Modifications

In one example, the self-aggregating polypeptide is an NSP2 protein with the amino acid sequence of SEQ ID NO:2. In another example, the self-aggregating polypeptide is an NSP2 protein with the amino acid sequence of SEQ ID NO:4. In other examples, the self-aggregating polypeptide is an NSP2 protein indicated in the preceding section by GENBANK® Accession Number. For example, the NSP2 protein of SEQ ID NO:2 can be encoded by the polynucleotide sequence of SEQ ID NO:1. Alternatively, the NSP2 protein of SEQ ID NO:2 can be encoded by a polynucleotide with one or more nucleotide substitutions, provided that the substitutions encode degenerate codons, that is, provided that the polynucleotide sequence does not alter the amino acid with respect to the reference NSP2 sequence. Likewise, the NSP2 protein of SEQ ID NO:4 can be encoded by the polynucleotide sequence of SEQ ID NO:3, or by a related polynucleotide sequence that differs from SEQ ID NO:3 solely by virtue of the degeneracy of the genetic code. Similarly, any of the NSP2 proteins disclosed by reference to a GENBANK® Accession No. can be encoded by any polynucleotide that encodes the particular NSP2 polypeptide. One of skill in the art will likewise recognize that any of the polynucleotide sequences described by reference to a GENBANK® Accession No. can be replaced by a related polynucleotide sequence that differs from the reference sequence due to the degeneracy of the genetic code.

In other embodiments, the self-aggregating polypeptide is an NSP2 protein with one or more conservative amino acid modifications (with respect to SEQ ID NO:2, SEQ ID NO:4 or any of the NSP2 proteins disclosed herein), for example, amino acid additions, deletions or substitutions as exemplified by the conservative amino acid substitutions provided in the definition section of this disclosure. A conservative amino acid modification, whether an addition, deletion or modification does not substantially alter the 3-dimensional structure of the polypeptide. For example, a conservative amino acid substitution does not disrupt the HIT-like fold or the ability of the NSP2 polypeptide to self-aggregate into stable multimers. For example, a suitable conservative amino acid substitution can alter (e.g., diminish) NTPase activity without affecting the structure of the HIT-like fold. For example, mutations can be made in any of the following amino acid positions (positions designated with respect to the SA11 NSP2 polypeptide): amino acid position 110, amino acid position 153, amino acid position 171, amino acid position 188, amino acid position 221, amino acid position 223, amino acid position 225 and amino acid position 227. For example, and one or more of the following specific amino acid substitutions can be made in an the NSP2 SA11 polypeptide: H110A, E153A, Y171A, K188A, H221A, K223A, H225A and/or R227A. Similarly, amino acid substitutions can be situated in regions other than the HIT-like fold. For example, amino acid substitutions can be situated at one or more of the following amino acid positions: amino acid position 37, amino acid position 38, amino acid position 59, amino acid position 60 and amino acid position 68. In specific examples, one or more of these amino acids is substituted with a glutamine without adversely affecting structure or function of the NSP2 polypeptide. For example, Additionally, non-conservative amino acid modifications of NSP2 polypeptides are known in the art, some of which may be suitable for use as antigen delivery platforms. For example, a temperature sensitive mutation in the simian NSP2 protein designated tsE (J. Virol. 76:7082-7093) is functionally impaired and forms large complexes in the presence of magnesium at non-permissive temperatures. Such a mutation is an example of a non-conservative amino acid modification. One of skill in the art can easily determine additional amino acid substitutions without undue experimentation. For example, suitable NSP2 polypeptides with conservative or non-conservative amino acid substitutions, deletions and/or additions possess the characteristic HIT-like fold and assemble into stable multimers that can be expressed and purified to acceptable yield and purity according to the methods described herein. Similarly, unsuitable NSP2 polypeptides can be distinguished from suitable NSP2 polypeptides, in that they have a disrupted structure (either within or outside the HIT-like fold) and do not assemble into stable multimeric ring structures. Such NSP2 polypeptides are typically difficult to express and/or purify and are less useful in the compositions disclosed herein.

NSP2 proteins having deletions of a small number of amino acids, for example, less than about 10% (e.g., less than about 8%, or less than about 5%, or less than about 2%, or less than about 1%) of the total number of amino acids in the wild type NSP2 protein are also favorably employed in the antigen delivery platform fusion proteins described herein. The deletion may be a terminal deletion, or an internal deletion, so long as the deletion does not substantially affect the structure or aggregation of the fusion protein. For example, in certain examples the NSP2 polypeptide is a fragment of a full-length NSP2 protein with a deletion of one or more amino acids from the C-terminus of the NSP2 protein. Typically, the number of deleted amino acids is small, such as no more than about 10 amino acids, for example, about seven amino acids, such as seven, or six, or five, or four, or three, or two, or a single amino acid. For example fragments of NSP2 polypeptides that lack up to about 10 amino acids (for example, that lack seven amino acids or fewer) are known to have an HIT-like fold and self-aggregate into stable ring like structures. Such fragments of full-length viral NSP2 proteins are NSP2 polypeptides suitable as components of the fusion proteins disclosed herein.

Additionally, the NSP2 protein component can include amino acid sequences in addition to those found in the protein in nature. The affinity tags discussed above are one example of an additional amino acid sequence. Other amino acid additions, such as multiple affinity tags, binding sites, or the like, can optionally be included so long as the additional amino acid sequences do not impair the ability of the fusion protein to be expressed or to form multimers that maintain the native structure.

### Polynucleotides Encoding Fusion Polypeptides

Nucleic acids encoding the fusion proteins (for example, monomeric fusion proteins) described herein are also provided. These nucleic acids include deoxyribonucleotides (DNA, cDNA) or ribodeoxynucleotides (RNA) sequences, or modified forms of either nucleotide, which encode the fusion polypeptides described herein. The term includes single and double stranded forms of DNA and/or RNA.

The nucleic acids that encode fusion polypeptides suitable as antigen delivery platforms include a polynucleotide sequence that encodes a self-aggregating viral NSP2 polypeptide. The polynucleotide sequence can encode any viral NSP2 polypeptide, or a functional fragment, variant, homolog or analog thereof, such as any of the NSP2 polypeptides, fragments, variants, homologs or analogs described in this disclosure. Accordingly, suitable polynucleotides encode a polypeptide with an HIT-like fold that self-assemble into stable multimeric ring structures. Numerous examples are provided above under the topical heading of “Fusion Polypeptides,” any of which are suitable as the basis for an epitope mounting platform.

In specific examples, the polynucleotide sequence encodes an NSP2 polypeptide represented by SEQ ID NO:2 or SEQ ID NO:4. For example, in one example described in detail in the examples, the nucleic acid that encodes the NSP2 polypeptide comprises the sequence represented by SEQ ID NO:1. In another example, the nucleic acid that encodes the NSP2 polypeptide comprises the sequence of SEQ ID NO:3. In other embodiments, the nucleic acid encoding the NSP2 polypeptide includes a polynucleotide sequence that encodes a polypeptide identical to that encoded by SEQ ID NO:1 or SEQ ID NO:3 (that is, SEQ ID NO:2 or SEQ ID NO:4, respectively), but that differs from one of these reference polynucleotides by virtue of the degeneracy of the genetic code.

In yet other embodiments, the nucleic acids that encode the NSP2 polypeptides differ more substantially from one or more reference sequences discussed herein. Nonetheless, provided that the structural and functional properties described above, namely the presence of an HIT-like fold and the ability to form multimeric ring structures, are maintained, such polynucleotide sequences are also suitable in the context of the antigen delivery platforms described herein. For example, numerous nucleic acids that share substantial sequence identity with the nucleic acids disclosed herein can be employed in fusion polypeptides suitable as antigen delivery platforms. Polynucleotides that encode NSP2 polypeptides of any rotavirus Groups A, B, C, D, E, F or G (or yet to be described rotavirus group) as well as more distantly related viral polypeptides can be used to in fusion polypeptides for use as an antigen delivery platform. For example, polynucleotide sequences that share as little as 30 or 40% sequence identity with SEQ ID NO:1 or SEQ ID NO:3 encode suitable NSP2 polypeptides. For example, SEQ ID NO:1 and SEQ ID NO:3, both of which are shown herein to encode NSP2 polypeptides suitable for use in the fusion polypeptides described share approximately 50% sequence identity. Nonetheless in some embodiments, polynucleotide sequences with more than 50% sequence identity, or more than 70%, or even more than 80%, 90%, or even greater sequence identity, such as 95%, 97% or 99% sequence identity to SEQ ID NO:1 or SEQ ID NO:3 or another sequence referenced herein are employed in the context of epitope mounting platforms.

The epitope mounting platforms described herein can include essentially any antigenic polypeptide. Accordingly, any polynucleotide sequence that encodes an antigenic polypeptide can be included as a component of the fusion polypeptides disclosed herein. For example, any polynucleotide sequence that encodes an exemplary antigenic polypeptide as described in the section above entitled “Fusion Polypeptides” is, of course, a suitable polynucleotide sequence in the context of the nucleic acids disclosed herein. In addition, numerous other polynucleotide sequences can be selected according to the particular application contemplated by the practitioner.

In certain examples described herein, the polynucleotide encoding the antigenic polypeptide are represented by SEQ ID NO:5, SEQ ID NO:7, SEQ ID NO:9 or polynucleotides having substantial sequence identity, (such as at least 75% or at least 80%, or at least 90%, or at least 95% or even 99% sequence identity) thereto. Similarly, while exemplary polynucleotides that encode linking peptides and affinity tags are provided herein, one of skill in the art will be able to select numerous additional polynucleotides that can be used in addition to or instead of the particular polynucleotides provided herein.

As will be appreciated by one of skill in the art, polynucleotide sequences that hybridize to any of the exemplary sequences disclosed herein, such as SEQ ID NO:1, SEQ ID NO:3, SEQ ID NO:5, SEQ ID NO:7 or SEQ ID NO:9. are also suitable in the context of the fusion polypeptides. Exemplary polynucleotides that encode fusion polypeptides include those described in SEQ ID NOs:15, 17 and 19. The amino acid sequences of exemplary fusion proteins encoded by these polynucleotide sequences are represented by SEQ ID NOs:16, 18, and 20, respectively.

The expression and purification of proteins, such as a monomeric NSP2 fusion protein, can be performed using standard laboratory techniques. Examples of such methods are discussed or referenced herein. After expression, purified proteins have many uses, including for instance functional analyses, antibody production, and diagnostics, as well as the prophylactic and therapeutic uses described below. Partial or full-length cDNA sequences, which encode the fusion proteins, may be ligated into bacterial expression vectors. Methods for expressing large amounts of protein from a cloned sequence introduced into Escherichia coli (E. coli) or baculovirus/Sf9 cells may be utilized for the purification, localization and functional analysis of proteins, as well as for the production of antibodies and vaccine compositions. For example, fusion proteins consisting of a self-aggregating polypeptide and an antigenic epitope can be used in various procedures, for instance to prepare polyclonal and monoclonal antibodies against these proteins. Thereafter, these antibodies may be used to purify proteins by immunoaffinity chromatography, in diagnostic assays to quantitate the levels of protein and to localize proteins in tissues and individual cells by immunofluorescence. More particularly, the fusion proteins and the polynucleotides encoding them described herein can be used to produce pharmaceutical compositions, including vaccine compositions suitable for prophylactic and/or therapeutic administration.

Methods and additional plasmid vectors for producing the polynucleotides encoding fusion proteins and for expressing these polynucleotides in bacterial and eukaryotic cells are well known in the art, and specific methods are described in Sambrook et al. (In Molecular Cloning: A Laboratory Manual, Ch. 17, CSHL, New York, 1989). Such fusion proteins may be made in large amounts, are easy to purify, and can be used to elicit an immune response, including an antibody response or a T cell response. Native proteins can be produced in bacteria by placing a strong, regulated promoter and an efficient ribosome-binding site upstream of the cloned gene. If low levels of protein are produced, additional steps may be taken to increase protein production; if high levels of protein are produced, purification is relatively easy. Suitable methods are presented in Sambrook et al. (In Molecular Cloning: A Laboratory Manual, CSHL, New York, 1989) and are well known in the art. Often, proteins expressed at high levels are found in insoluble inclusion bodies. Methods for extracting proteins from these aggregates are described by Sambrook et al. (In Molecular Cloning. A Laboratory Manual, Ch. 17, CSHL, New York, 1989).

Proteins, including fusion proteins, may be isolated from protein gels, lyophilized, ground into a powder and used as an antigen.

Vector systems suitable for the expression of polynucleotides encoding fusion proteins include, in addition to the specific vectors described in the examples, the pUR series of vectors (Ruther and Muller-Hill, EMBO J. 2:1791, 1983), pEX1-3 (Stanley and Luzio, EMBO J. 3:1429, 1984) and pMR100 (Gray et al., Proc. Natl. Acad. Sci. USA 79:6598, 1982). Vectors suitable for the production of intact native proteins include pKC30 (Shimatake and Rosenberg, Nature 292:128, 1981), pKK177-3 (Amann and Brosius, Gene 40:183, 1985) and pET-3 (Studiar and Moffatt, J. Mol. Biol. 189:113, 1986), as well as the IPTG inducible expression vector pQE60 described in the Examples.

The DNA sequence can also be transferred from its existing context to other cloning vehicles, such as other plasmids, bacteriophages, cosmids, animal viruses and yeast artificial chromosomes (YACs) (Burke et al., Science 236:806-812, 1987). These vectors may then be introduced into a variety of hosts including somatic cells, and simple or complex organisms, such as bacteria, fungi (Timberlake and Marshall, Science 244:1313-1317, 1989), invertebrates, plants (Gasser and Fraley, Science 244:1293, 1989), and animals (Pursel et al., Science 244:1281-1288, 1989), which cell or organisms are rendered transgenic by the introduction of the heterologous cDNA.

For expression in mammalian cells, a cDNA sequence may be ligated to heterologous promoters, such as the simian virus (SV) 40 promoter in the pSV2 vector (Mulligan and Berg, Proc. Natl. Acad. Sci. USA 78:2072-2076, 1981), and introduced into cells, such as monkey COS-1 cells (Gluzman, Cell 23:175-182, 1981), to achieve transient or long-term expression. The stable integration of the chimeric gene construct may be maintained in mammalian cells by biochemical selection, such as neomycin (Southern and Berg, J. Mol. Appl. Genet. 1:327-341, 1982) and mycophenolic acid (Mulligan and Berg, Proc. Natl. Acad. Sci. USA 78:2072-2076, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR or other in vitro amplification.

A cDNA sequence (or portions derived from it) such as a cDNA encoding an antigenic polypeptide or a fusion protein can be introduced into eukaryotic expression vectors by conventional techniques. These vectors are designed to permit the transcription of the cDNA in eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan et al., Proc. Natl. Acad. Sci. USA 78:1078-2076, 1981; Gorman et al., Proc. Natl. Acad. Sci USA 78:6777-6781, 1982). The level of expression of the cDNA can be manipulated with this type of vector, either by using promoters that have different activities (for example, the baculovirus pAC373 can express cDNAs at high levels in S. frugiperda cells (Summers and Smith, In Genetically Altered Viruses and the Environment, Fields et al. (Eds.) 22:319-328, CSHL Press, Cold Spring Harbor, N.Y., 1985) or by using vectors that contain promoters amenable to modulation, for example, the glucocorticoid-responsive promoter from the mouse mammary tumor virus (Lee et al., Nature 294:228, 1982). The expression of the cDNA can be monitored in the recipient cells 24 to 72 hours after introduction (transient expression).

In addition, some vectors contain selectable markers such as the gpt (Mulligan and Berg, Proc. Natl. Acad. Sci. USA 78:2072-2076, 1981) or neo (Southern and Berg, J. Mol. Appl. Genet. 1:327-341, 1982) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., Mol. Cell Biol. 1:486, 1981) or Epstein-Barr (Sugden et al., Mol. Cell Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., J. Biol. Chem. 253:1357, 1978).

The transfer of DNA into eukaryotic, in particular human or other mammalian cells, is now a conventional technique. The vectors are introduced into the recipient cells as pure DNA (transfection) by, for example, precipitation with calcium phosphate (Graham and vander Eb, Virology 52:466, 1973) or strontium phosphate (Brash et al., Mol. Cell Biol. 7:2013, 1987), electroporation (Neumann et al., EMBO J 1:841, 1982), lipofection (Felgner et al., Proc. Natl. Acad. Sci USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-2167, 1980), or pellet guns (Klein et al., Nature 327:70, 1987). Alternatively, the cDNA, or fragments thereof, can be introduced by infection with virus vectors. Systems are developed that use, for example, retroviruses (Bernstein et al., Gen. Engr'g 7:235, 1985), adenoviruses (Ahmad et al., J. Virol. 57:267, 1986), or Herpes virus (Spaete et al., Cell 30:295, 1982). Polynucleotides that encode proteins, such as fusion proteins, can also be delivered to target cells in vitro via non-infectious systems, for instance liposomes.

Using the above techniques, the expression vectors containing a polynucleotide encoding a monomeric fusion protein as described herein or cDNA, or fragments or variants or mutants thereof, can be introduced into human cells, mammalian cells from other species or non-mammalian cells as desired. The choice of cell is determined by the purpose of the treatment. For example, monkey COS cells (Gluzman, Cell 23:175-182, 1981) that produce high levels of the SV40 T antigen and permit the replication of vectors containing the SV40 origin of replication may be used. Similarly, Chinese hamster ovary (CHO), mouse NIH 3T3 fibroblasts or human fibroblasts or lymphoblasts may be used.

The present disclosure, thus, encompasses recombinant vectors that comprise all or part of the polynucleotides encoding self-aggregating fusion proteins or cDNA sequences, for expression in a suitable host, either alone or as a labeled or otherwise detectable protein. The DNA is operatively linked in the vector to an expression control sequence in the recombinant DNA molecule so that the fusion polypeptide or protein can be expressed. The expression control sequence may be selected from the group consisting of sequences that control the expression of genes of prokaryotic or eukaryotic cells and their viruses and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

The host cell, which may be transfected with the vector of this disclosure, may be selected from the group consisting of E. coli, Pseudomonas, Bacillus subtilis, Bacillus stearothermophilus or other bacilli; other bacteria; yeast; fungi; insect; mouse or other animal; plant hosts; or human tissue cells.

### Recovery of Multimeric Fusion Protein Ring Structures

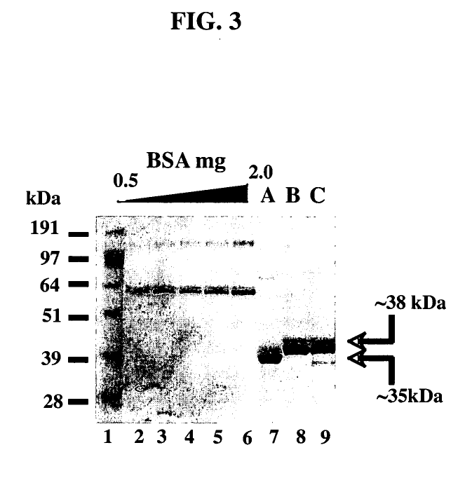

Multimeric fusion protein ring structures can be recovered for administration to a subject in a vaccine composition (or for other purposes) using any of a variety of methods known in the art for the purification of recombinant polypeptides. As illustrated in FIGS. 3, 4A and 5A, the fusion proteins disclosed herein are produced efficiently by transfected cells and can be recovered in quantity using a simple nickel (NTA-agarose) affinity chromatography purification procedure.

A variety of common methods of protein purification may be used to purify the disclosed fusion proteins. Such methods include, for instance, protein chromatographic methods including ion exchange, gel filtration, HPLC, monoclonal antibody affinity chromatography and isolation of insoluble protein inclusion bodies after over production. As described in further detail in the examples, in a favorable embodiment one or more purification affinity-tags, for instance a six-histidine sequence, is recombinantly fused to the protein and used to facilitate polypeptide purification (optionally, in addition to another functionalizing portion of the fusion, such as a targeting domain or another tag, or a fluorescent protein, peptide, or other marker).

A specific proteolytic site, for instance a thrombin-specific digestion site, can be engineered into the protein between the tag and the remainder of the fusion to facilitate removal of the tag after purification, if such removal is desired.

Commercially produced protein expression/purification kits provide tailored protocols for the purification of proteins made using each system. See, for instance, the QIAEXPRESS™ expression system from QIAGEN (Chatsworth, Calif.) and various expression systems provided by INVITROGEN (Carlsbad, Calif.). Where a commercial kit is employed to produce an APOBEC3G fusion protein, the manufacturer's purification protocol is a preferred protocol for purification of that protein. For instance, proteins expressed with an amino-terminal hexa-histidine tag can be purified by binding to nickel-nitrilotriacetic acid (Ni-NTA) metal affinity chromatography matrix (The QIA expressionist, QIAGEN, 1997).

More generally, the binding specificities of the NSP2 component or the antigenic epitope, or both, of a fusion protein can be exploited to facilitate specific purification of the proteins. One example method of performing such specific purification would be column chromatography using column resin to which the an antibody, target molecule, or an appropriate epitope or fragment or domain of the antibody or target molecule, has been attached.

In addition to protein expression and purification guidelines provided herein, protein expression/purification kits are produced commercially. See, for instance, the QIAEXPRESS™ expression system from QIAGEN (Chatsworth, Calif.) and various expression systems provided by INVITROGEN (Carlsbad, Calif.). Such kits can be used for production and purification of the fusion proteins described herein.

### Therapeutic Methods and Pharmaceutical Compositions

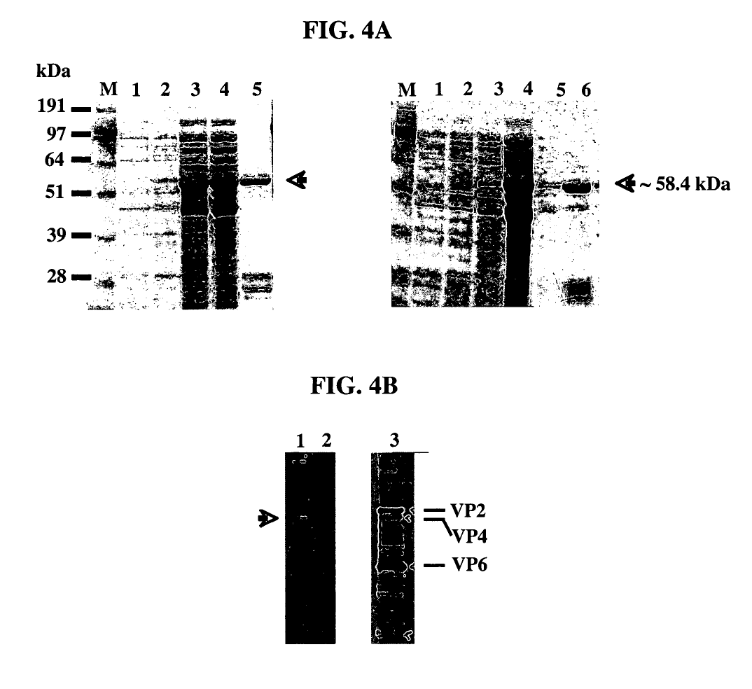

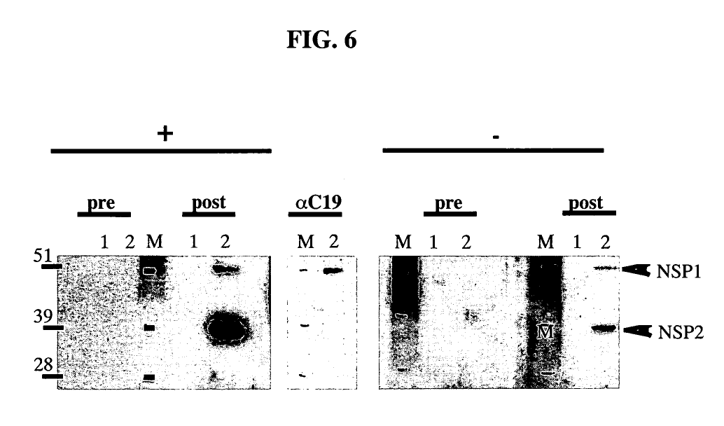

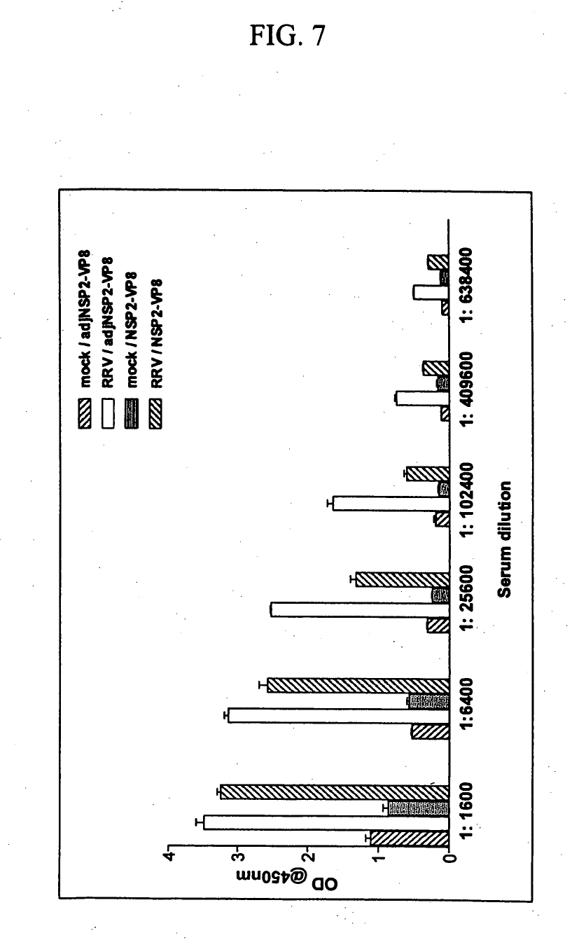

Polynucleotides encoding the self-aggregating fusion proteins, and the stable multimeric ring structures formed by polypeptides expressed from such polynucleotides can be administered to a subject in order to generate an immune response against an antigenic polypeptide component of the fusion protein. Typically, an immune response includes the production of antibodies that interact specifically with an epitope of the antigenic polypeptide. Illustrative examples of a specific antibody response against an antigenic polypeptide presented in the context of the antigen delivery platform fusion proteins described herein are shown in FIGS. 4B and 6. The fusion proteins described herein are capable of eliciting a high titer of serum antibodies when administered with or without adjuvant as illustrated in FIG. 7 and Table 1.

A therapeutically effective amount of any one or more of these immunogenic fusion proteins, or a polynucleotide encoding one or more of these polypeptides as disclosed above (or complexes formed by aggregation of such monomeric fusion proteins), can be administered to a subject to prevent, inhibit or to treat a condition, symptom or disease, such as a disease resulting from exposure to a pathogenic organism. In one example, stable ring structures formed by monomeric fusion protein subunits are administered. In another example, one or more polynucleotides encoding at least one fusion polypeptide are administered. As such, the fusion polypeptides and polynucleotides encoding fusion polypeptides can be administered as vaccines to prophylactically or therapeutically induce or enhance an immune response. For example, the pharmaceutical compositions described herein can be administered to stimulate a protective immune response against a pathogenic organism, such as a pathogenic virus.

A single administration can be utilized to prevent or treat a condition, symptom or disease, or multiple sequential administrations can be performed. In another example, more than one of the fusion polypeptides (or multiple polynucleotides encoding the polypeptides), including different antigenic epitopes are administered. The polypeptides or polynucleotides can be administered simultaneously, or sequentially.

In exemplary applications, compositions are administered to a subject suffering from a disease, or likely to be exposed to an infection, by a pathogenic organism, such as a pathogenic virus, in an amount sufficient to raise an immune response to the pathogen. Administration induces a sufficient immune response to reduce pathogen load, to prevent or lessen a later infection with the pathogen, or to reduce a sign or a symptom of infection. Amounts effective for this use will depend upon the nature of the pathogen, the general state of the subject's health, and the robustness of the subject's immune system. A therapeutically effective amount of the compound is that which provides either subjective relief of a symptom(s), an objectively identifiable improvement as noted by the clinician or other qualified observer, or inhibit development of symptoms associated with infection.

The aggregated fusion polypeptides and polynucleotides encoding them can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, Pa., 1995) such as by intramuscular, subcutaneous, or intravenous injection, but even oral, nasal, or anal administration is contemplated. Commonly, the fusion polypeptides or polynucleotides are administered in a formulation including a carrier or excipient. A wide variety of suitable excipients are known in the art, including physiological saline, PBS and the like. Optionally, the formulation includes additional components, such as aluminum hydroxylphophosulfate, alum, diphtheria CRM₁₉₇, or liposomes. In one embodiment, administration is by subcutaneous or intramuscular injection. To extend the time during which the peptide or protein is available to stimulate a response, the peptide or protein can be provided as an implant, an oily injection, or as a particulate system. The particulate system can be a microparticle, a microcapsule, a microsphere, a nanocapsule, or similar particle. (see, e.g., Banga, supra). A particulate carrier based on a synthetic polymer has been shown to act as an adjuvant to enhance the immune response, in addition to providing a controlled release. Aluminum salts may also be used as adjuvants to produce an immune response.

In one specific, non-limiting example, fusion polypeptides described herein are administered in a manner to direct the immune response to a cellular response (that is, a cytotoxic T lymphocyte (CTL) response), rather than, or in addition to, a humoral (antibody) response. A number of means for inducing cellular responses, both in vitro and in vivo, are known. Lipids have been identified as agents capable of assisting in priming CTL in vivo against various antigens. For example, as described in U.S. Pat. No. 5,662,907, palmitic acid residues can be attached to the alpha and epsilon amino groups of a lysine residue and then linked (e.g., via one or more linking residues, such as glycine, glycine-glycine, serine, serine-serine, or the like) to an immunogenic peptide. The lipidated peptide can then be injected directly in a micellar form, incorporated in a liposome, or emulsified in an adjuvant. As another example, E. coli lipoproteins, such as tripalmitoyl-S-glycerylcysteinlyseryl-serine can be used to prime tumor specific CTL when covalently attached to an appropriate peptide (see, Deres et al., Nature 342:561, 1989). Further, as the induction of neutralizing antibodies can also be primed with the same molecule conjugated to a peptide which displays an appropriate epitope, two compositions can be combined to elicit both humoral and cell-mediated responses where that is deemed desirable.

In yet another embodiment, to induce a CTL response to an NSP2 fusion polypeptide, a MHC Class II-restricted T-helper epitope is added to the antigenic polypeptide to induce T-helper cells to secrete cytokines in the microenvironment to activate CTL precursor cells. The technique further involves adding short lipid molecules to retain the construct at the site of the injection for several days to localize the antigen at the site of the injection and enhance its proximity to dendritic cells or other “professional” antigen presenting cells over a period of time (see Chesnut et al., “Design and Testing of Peptide-Based Cytotoxic T-Cell-Mediated Immunotherapeutics to Treat Infectious Diseases and Cancer,” in Powell et al., eds., Vaccine Design, the Subunit and Adjuvant Approach, Plenum Press, New York, 1995).

A pharmaceutical composition including one or more antigen presenting fusion proteins is thus provided. In one embodiment, the NSP2 fusion protein is mixed with an adjuvant containing two or more of a stabilizing detergent, a micelle-forming agent, and an oil. Suitable stabilizing detergents, micelle-forming agents, and oils are detailed in U.S. Pat. No. 5,585,103; U.S. Pat. No. 5,709,860; U.S. Pat. No. 5,270,202; and U.S. Pat. No. 5,695,770, all of which are incorporated by reference. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20™, TWEEN 60™, ZWITTERGENT™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, for example, Schmolka, J. Am. Oil. Chem. Soc. 54:110, 1977; and Hunter et al., J. Immunol 129:1244, 1981, PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, or in an amount between 1.25 and 5%.

The oil included in the composition is chosen to promote the retention of the antigen in oil-in-water emulsion, such as to provide a vehicle for the desired antigen, and preferably has a melting temperature of less than 65° C. such that emulsion is formed either at room temperature (about 20° C. to 25° C.), or once the temperature of the emulsion is brought down to room temperature. Examples of such oils include squalene, Squalane, EICOSANE™, tetratetracontane, glycerol, and peanut oil or other vegetable oils. In one specific, non-limiting example, the oil is provided in an amount between 1 and 10%, or between 2.5 and 5%. The oil should be both biodegradable and biocompatible so that the body can break down the oil over time, and so that no adverse affects, such as granulomas, are evident upon use of the oil.

An adjuvant can be included in the composition. In one example, the adjuvant is a water-in-oil emulsion in which antigen solution is emulsified in mineral oil (such as Freund's incomplete adjuvant or montanide-ISA). In one embodiment, the adjuvant is a mixture of stabilizing detergents, micelle-forming agent, and oil available under the name PROVAX® (IDEC Pharmaceuticals, San Diego, Calif.).

In another embodiment, a pharmaceutical composition includes a nucleic acid encoding one or more NSP2-fusion protein polypeptides as disclosed herein. A therapeutically effective amount of the immunogenic polynucleotide can be administered to a subject in order to generate an immune response.

One approach to administration of nucleic acids is direct immunization with plasmid DNA, such as with a mammalian expression plasmid. As described above, the nucleotide sequence encoding an NSP2-fusion protein can be placed under the control of a promoter to increase expression of the molecule. Suitable vectors are described, for example, in U.S. Pat. No. 6,562,376.

Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. No. 5,593,972 and U.S. Pat. No. 5,817,637 (which describe operatively linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids, and immune-stimulating constructs, or ISCOMS™, negatively charged cage-like structures of 30-40 nm in size formed spontaneously on mixing cholesterol and QUIL A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In another approach to using nucleic acids for immunization, an NSP2-fusion polypeptide can also be expressed by an attenuated viral host or vector, or a bacterial vector. Recombinant adeno-associated virus (AAV), herpes virus, retrovirus, or other viral vectors can be used to express the peptide or protein, thereby eliciting a CTL response.

In one embodiment, a nucleic acid encoding an NSP2-fusion protein is introduced directly into cells. For example, the nucleic acid may be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites, including tissues subject to or in proximity to a site of infection. Dosages for injection are usually around 0.5 μg/kg to about 50 mg/kg, and typically are about 0.005 mg/kg to about 5 mg/kg (see, e.g., U.S. Pat. No. 5,589,466).

In one specific, non-limiting example, a pharmaceutical composition for intravenous administration, would include about 0.1 μg to 10 mg of NSP2-fusion protein per subject per day. Dosages from 0.1 pg to about 100 mg per subject per day can be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remingtons Pharmaceuticals Sciences, 19^{th} Ed., Mack Publishing Company, Easton, Pa. (1995).

The compositions can be administered for therapeutic treatments. In therapeutic applications, a therapeutically effective amount of the composition is administered to a subject suffering from a disease, such as a disease resulting from infection by a pathogenic organism, such as a pathogenic virus. Single or multiple administrations of the compositions are administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

Controlled release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, Pa. (1995). Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, N.Y., pp. 219-342 (1994); Tice & Tabibi, Treatise on Controlled Drug Delivery, A. Kydonieus, ed., Marcel Dekker, Inc. New York, N.Y., pp. 315-339 (1992)).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

### Immunodiagnostic Reagents and Kits

In addition to the therapeutic methods provided above, any of the NSP2 fusion proteins disclosed herein can be utilized to produce antigen specific immunodiagnostic reagents, for example, for serosurveillance. Peptides presented in the context of an NSP2 fusion polypeptide possess a greater freedom of movement, and therefore, greater accessibility to antibody and ligands that peptides directly bound to a substrate (for example, as in common ELISA procedures). This provides increased sensitivity without a loss of specificity when the fusion polypeptide is employed in an enzyme-based immunoassay (“EIA”). Immunodiagnostic reagents can be designed from any of the antigenic polypeptide described herein. For example, the presence of serum antibodies to such important viral pathogens as HIV, SARS, and Foot and Mouth Disease (“FMD”) can be monitored using NSP2 fusion polypeptides including as an antigenic polypeptide component HIV gp41/env/gag, the SARS coronavirus immunodominant spike (“S”), membrane (“M”), and nucleocapsid (“N”) peptides, and the FMD 3B nonstructural protein, respectively. Similarly, serum antibodies to such viruses as the Norwalk virus can be detected using an NSP2 fusion polypeptide incorporating, e.g., the Norwalk virus P2 epitope.

Additionally, any such immunodiagnostic reagents can be provided as components of a kit. Optionally, such a kit includes additional components including packaging, instructions and various other reagents, such as buffers, substrates, antibodies or ligands, such as control antibodies or ligands, and detection reagents.

The disclosure is illustrated by the following non-limiting Examples.

### EXAMPLES

### Example I

### Construction of the NSP2 Antigen Delivery Vector

NSP2 expression vectors were constructed as previously described (Taraporewala et al., J. Virol. (1999) 73:9934-9943, using routine molecular biology techniques (see, e.g., Sambrook et al. (1989) Molecular Cloning: A Laboratory Manual, Ch. 17, CSHL, New York).

In one example, a cDNA containing the NSP2 open reading frame (“ORF”) of SA11 rotavirus was cloned into the IPTG-inducible bacterial expression vector pQE60 (SEQ ID NO:37), such that the recombinant protein contained a C-terminal tag of six His residues. Gene 8 was amplified from pSP65g8 by using the Ampli Taq system (Life Technologies) and the forward primer 5′-CCGAAACCATGGCTGAGCTAG-3′ (SEQ ID NO:25; NcoI site is underlined) and the reverse primer 5′-CGGAGATCTACGCCAACTTGAGAAAC-3′ (SEQ ID NO:26; Bg/II site is underlined). The amplification conditions were as follows: 94° C. for 5 min, 1 cycle; 94° C. for 1 min, 37° C. for 1 min, and 72° C. for 2.5 min, 10 cycles; 94° C. for 1 min, 45° C. for 1 min, and 72° C. for 2.5 min, 20 cycles. The 998-bp product was gel purified and ligated into the PCR cloning vector pT7Blue (Novagen). Following transformation of Escherichia coli DH5, bacteria containing the appropriate plasmid (pT7Bg8) were identified based on antibiotic resistance, plasmid size, and restriction enzyme digestion. The sequence accuracy of the gene 8 insert in pT7B-g8 was confirmed by automated sequencing with an ABI PRISM 310 genetic analyzer (PE Applied Biosystems). To express rNSP2, pT7Bg8 was digested with NcoI and Bg/II, and the gene 8 fragment was ligated into the isopropyl-β-D-thiogalactopyranoside (IPTG)-inducible expression vector pQE60 (Qiagen), similarly digested with NcoI and Bg/II. Following transformation into E. coli DH5α, bacteria with the appropriate plasmid (pQE60g8) were identified and the plasmid was purified. pQE60g8 was then electroporated into E. coli M15 carrying the pREP4 repressor plasmid. Appropriate transformants were identified based on antibiotic resistance, restriction enzyme digestion, and expression of rNSP2. In pQE60g8, the ORF for NSP2 is situated two amino acids upstream (arginine-serine encoded by the in frame Bg/II recognition sequence) from six in-frame codons for histidine (“His”). Thus, rNSP2 expressed from pQE60g8 is tagged at its C terminus with six histidine (“His₆”) residues.

To demonstrate that rotavirus NSP2 expression vectors and fusion proteins represented a generalizable platform in which alternative NSP2 polypeptides can be employed with favorable results, an alternative NSP2 epitope mounting platform was characterized. Gene 9 of the Group C strain Bristol virus was amplified from seg9/M13 using the Pfu Turbo DNA polymerase (Stratagene) and the forward primer 5′-CTGGTGCCATGGCCGA-3′ (SEQ ID NO:31; NcoI site underlined) and the reverse primer 5′-CACTACAAGATCTATTTCCAACCATAG-3′ (SEQ ID NO:32; BglII site underlined). The amplification conditions were as follows: 94 C for 4 min, 1 cycle; 94 C for 30 sec, 45 C for 30 sec, and 68 C for 1.5 min, 30 cycles; and 68 C for 6 min, 1 cycle. The 938-bp product was digested with Nco I and Bgl II, gel purified and ligated into the IPTG-inducible expression vector pQE60 (Qiagen), that was similarly digested with NcoI and BglII. Following transformation into E.coli DH5α, bacteria with the appropriate plasmid (pQE60g9C) were identified and the sequence accuracy was confirmed by automated sequencing with a ABI PRISM 3100 genetic analyzer (PE Applied Biosystems). The correct plasmid was then electroporated into E.coli M15. Appropriate tranformants were identified based on antibiotic resistance, restriction enzyme digestion, and expression of rNSP2. In pQE60g9C, the ORF for NSP2 is situated two amino acids upstream (arginine-serine encoded by the in frame BglII recognition sequence) from in-frame codons for histidine. Thus, rNSP2 expressed from pQE60g9 is tagged at its C terminus with six histidine residues. The nucleotide sequence encoding the histidine tagged Bristol NSP2 polypeptide is provided in SEQ ID NO:23, the amino acid of the polypeptide is shown in SEQ ID NO:24.

Not I sites were introduced in SA11 NSP2 and Bristol NSP2 by outward PCR using the following templates, pQE60g8 and pQE60g9C, respectively, and the primer pair 5′-GAGCGGCCGCTCGGGCCTGGTCCGTGATGGTG-3′ (SEQ ID NO:33); 5′-GAAGCGGCCGCGGACCAGGCCCGTCTCATCACCATCACCATCACTAAGCTTAATTAGCTGAGCTTGGACTCCTGTTGATAGATCC-3′ (SEQ ID NO:34) and 5′-CCGCGGCCGCTCGGGCCTGGTCCGTGATGGTGATGGTGATGAGATCT-3′ (SEQ ID NO:35) and 5′-CCGCGGCCGCTCGGGCCTGGTCCGTGATGGTGATGGTGATGATGAGATCT-3′ (SEQ ID NO:36), respectively. The amplification was carried out using the Expand High Fidelity amplification system (Roche) and the following conditions: 94° C. for 3 min, 94° C. for 1 min, 37° C. for 1 min, 68° C. for 6 min, 10 cycles; 94° C. for 1 min, 42° C. for 1 min, 68° C. for 6 min, 20 cycles. Each of the PCR products was gel-purified, treated with T4-polynucleotide kinase (NEB) and self-ligated with T4-ligase (NEB) followed by transformation into competent E. coli DH5α. Appropriate transformants identified based on antibiotic resistance, restriction enzyme digestion and sequencing. To express the NSP2 EMPs (both, SA11 and Bristol), the pQE60g8/NotI and pQE60g9c/NotI were electroporated into E. coli M15 cells carrying the pREP4 repressor plasmid. This allowed for the construction of a vectors with SA11 and Bristol NSP2 with a NotI site for the insertion of a selected antigenic epitope.

### Example 2

### Construction of Exemplary NSP2 Fusion Proteins

Several exemplary NSP2 fusion proteins were constructed to demonstrate their efficacy as an antigen delivery platform. Using the pQE60g8 plasmid described in Example 1, polynucleotides encoding the desired antigenic epitopes were operatively linked to the NSP2 polypeptide via a short flexible linker sequence. That is, the polynucleotide encoding the desired antigenic polypeptide was inserted into the NSP2 expression plasmid such that the multiple components of the fusion protein were expressed as a contiguous polypeptide in order to maintain the desired open reading frames in the N-terminal to C-terminal direction: NSP2 polypeptide; GPGP linker; antigenic polypeptide. In different embodiments, the NSP2 expression plasmid included an internal or terminal poly-histidine tag.

NSP2-NSP1.RRV.C19

Nineteen amino acid residues from the C-terminus of the RRV strain NSP1 were joined in frame with the pQE60g8 NSP2-(His₆) polypeptide by PCR-mediated modification. The PCR reaction mixture included the forward primer: 5′-CTTCTGATCTCGGACTCTGAAGATGACGATTAAGCTTAATTAGCTGAGCTTGGACTCCTG-3′ (SEQ ID NO:27) and 5′-CTCATATTCTTCAGATAGTTTTCCTTCCGGGCCTGGTCCGTGATGGTGATGGTGATGAGATCTAACGCC-3′ (SEQ ID NO:28) and a combination of Taq and Pwo DNA polymerases. The amplified DNA was blunt-ended with T4 DNA polymerase, treated with T4 DNa polynucleotide kinase and self-ligated with T4 ligase. Following transformation into E. coli DH5α, positive clones were selected based on antibiotic resistance and sequencing. The polynucleotide sequence encoding the NSP2-NSP1.RRV.C19 fusion polypeptide is provided in its entirety as SEQ ID NO:15. The 317 amino acid SA11 NSP2 ORF (SEQ ID NO:2) is encoded by polynucleotides 1 through 951 (SEQ ID NO:1), followed by a poly-histidine tag (positions 958-975; SEQ ID NO: 11) and a GPGP linker (positions 976-987; SEQ ID NO:13). The BglII site used to construct the pQE60g8 plasmid occupies nucleotide positions 952-957. The antigenic epitope of RRV NSP1 is encoded by the nucleotide residues 988-1047 (SEQ ID NO:5). The amino acid sequence of the encoded NSP2 protein is indicated in SEQ ID NO:2. The poly-histidine tag is represented by SEQ ID NO:12 and the GPGP linker is represented by SEQ ID NO:14. The RRV NSP1.C19 amino acid sequence is shown in SEQ ID NO:6.

NSP2-NSP1.SA11.C19

Similarly, a polypeptide from the C-terminus of the NSP1 protein of the rotavirus SA11 strain was joined in frame with the pQE60g8 NSP2-(His₆) polypeptide via a four amino acid linker sequence (glycine-proline-glycine-proline). The PCR reaction mixture included the forward primer: 5′-CTACTGATCTCCAACTCAGAAGATGACAATGAGTAAGCTTAATTAGCTGAGCTTGGACTCCTG-3′ (SEQ ID NO:29) and 5′-CTCAAATTCTTCAGTTAAAGTTCCAGACGGGCCTGGTCCGTGATGGTGATGGTGATGAGATCTAACGCC-3′ (SEQ ID NO:30) and a combination of Taq and Pwo DNA polymerases. The amplified DNA was blunt-ended with T4 DNA polymerase, treated with T4 DNA polynucleotide kinase and self-ligated with T4 ligase. The polynucleotide sequence encoding the NSP2-NSP1.SA11.C19 fusion protein is represented by SEQ ID NO:17. The 317 amino acid NSP2 ORF is encoded by polynucleotides 1 through 951, followed by a poly-histidine tag and a GPGP linker (positions 958-975 and 976-987, respectively). The C19 antigenic polypeptide of SA11 NSP1 (SEQ ID NO:8) is encoded by nucleotide residues 988-1047 (SEQ ID NO:7).

NSP2-VP8

A polynucleotide sequence encoding the VP4 fragment of the VP8 protein of the rotavirus RRV strain was operatively linked to the NSP2-(His₆) encoding sequence of pQE60g8. The polynucleotide sequence encoding the NSP2-VP8 fusion polypeptide is provided in its entirety as SEQ ID NO:19. As described above, the NSP2 ORF is encoded by polynucleotides 1-951. The VP8 ORF (SEQ ID NO:10), extending from positions 1000 to 1494 (SEQ ID NO:9), was inserted in frame at a NotI site. The reconstructed NotI sites occupy positions 988-996 and 1495-1503 at the 5′ and 3′ ends of the VP8 sequence, respectively. Additionally, a second (His₆) tag was placed at the C-terminus of the fusion protein adjacent to a GPGP(S) linker (nucleotide positions 1504-1536).

NSP2-VP8 (GGS)

In another example, the GPGP hinge connecting the SA11 NSP2 polypeptide and an RRV VP antigenic polypeptide was substituted by an alternative linear linking peptide with the amino acid sequence GGS. This construct was further modified by the elimination of the internal poly-histidine tag. As described above, a polynucleotide sequence encoding the VP4 fragment of the VP8 protein was operatively linked to a polynucleotide sequence encoding the SA11 NSP2 polypeptide in the pQE60 vector backbone. The protein encoding sequences are joined via a short polynucleotide encoding a peptide with the amino acid sequence glycine-glycine-serine rather than the previously described GPGP linking peptide. The polynucleotide sequence encoding the NSP2-VP8 fusion polypeptide is represented by SEQ ID NO:21. The NSP2 polypeptide is encoded by nucleotides 1-951; the GGS linear linking sequence is encoded by nucleotides 952-960; and the VP8 antigen is encoded by nucleotides 961-1449. A polynucleotide sequence encoding an additional linking sequence followed by a poly-histidine tag was placed at the 3′ end of the nucleic acid encoding the fusion polypeptide (nucleotides 1450-1506). The NSP2-VP8 fusion polypeptide is represented by SEQ ID NO:22.

### Example 3

### Expression and Purification of rNSP2-Fusion Proteins

The recombinant NSP2 fusion proteins described herein can be effectively and efficiently recovered under non-denaturing conditions to yield structurally intact multimeric ring structures. For example, one liter of bacterial culture yields up to 8-10 mg of highly soluble protein that can be rendered to greater than 90% homogeneity with a single-step affinity chromatography using Ni-NTA columns.

In brief, E. coli M15[pREP4] containing a fusion protein vector were grown to an optical density of 0.5 at 600 nm in Terrific Broth (Quality Biologics), and the expression of the NSP2-fusion protein construct was induced by adding IPTG to a final concentration of 1 mM. After incubation for 4 to 5 h at 37° C., the bacteria were recovered by centrifugation at 4,000× g for 10 min, and the His₆-tagged rNSP2-fusion protein was purified under native conditions on a Ni-nitrilotriacetic acid (NTA) agarose column (Qiagen) according to the manufacturer's protocols. The final eluate was dialyzed against low-salt buffer (LSB; 2 mM Tris-HCl [pH 7.2], 0.5 mM EDTA, 0.5 mM dithiothreitol [DTT]) for 48 h at 4° C. The concentration of the purified protein was determined by Bradford assay using bovine serum albumin (“BSA”) as the protein standard and by comparison with known amounts of BSA coelectrophoresed on sodium dodecyl sulfate (SDS)-polyacrylamide gels and Coomassie blue staining. Purified rNSP2-fusion proteins were adjusted to a concentration of 0.5 mg per ml and stored at 4° C.

FIG. 3 shows an exemplary SDS-PAGE evaluation of NSP2-NSP1.C19 fusion proteins. Expression of NSP2-NSP1.C19 fusion proteins was induced with IPTG in transformed bacterial cells. After purification over NTA-agarose as described above, the fusion protein eluates were separated by SDS-PAGE and stained with Coomassie blue. Lane 1 contains molecular weight markers. Lanes 2-5 contain increasing concentrations of BSA. Lane 7 contains NSP2 protein and lanes 8 and 9 are NSP2-NSP1.C19 fusion proteins, wherein the NSP1.C19 fusion partner is derived from the SA11 and RRV viruses, respectively. As can be seen in lanes 8 and 9, the fusion protein is produced in quantities comparable to NSP2, and purifies to near homogeneity with a single passage over NTA agarose.

Similarly, NSP2-VP8 fusion proteins were produced and purified as described above. FIG. 4A illustrates the sequential purification of NSP2-VP8 fusion protein expressed in bacterial cells. The left panel illustrates production and purification of an NSP2-VP8 fusion polypeptide that includes the SA11 NSP2 polypeptide and the RRV VP8 antigenic VP4 polypeptide joined via a GPGP linking peptide that includes both poly-histidine tags flanking the VP8 antigen. The right panel illustrates production and purification of an NSP2-VP8 fusion polypeptide that includes the RRV-VP8 polypeptide joined to the SA11 NSP2 polypeptide via a GGS linking peptide and tagged at the C-terminus with a poly-histidine tag. The lanes are designated as follows: M: molecular weight standards; lane 1: lysate from uninduced cells; lane 2: lysate from cells induced with 1 mM IPTG; lane 3: soluble fraction of the induced cell lysate; lane 4: flow through fraction following NTA-agarose column chromatography; lanes 5 and 6 (right panel only): eluate after dialysis of nickel-bound fraction. Substantial enrichment of the NSP2-VP8 fusion proteins was achieved after a single passage over NTA-agarose, as seen in lane 5. Additional quantitative recovery was obtained by repeating the elution step.

### Example 4

### Production and Purification of a Group C NSP2 Octamer

Group C (Bristol) NSP2 octamers were expressed and purified essentially as described above with respect to the group A (SA11) octamers described above, with minor modifications to the procedure. These procedural modifications can be used interchangeably regardless of the strain from which the NSP2 polypeptide was derived.

In brief, E. coli, strain SG13009 (QIAGEN™) was transfected with a plasmid vector that included the Bristol strain NSP2 polypeptide (NSP2c) tagged with a poly-histidine sequence (His₆) at its C-terminus in the pQE60 plasmid, as described above (that is a plasmid vector containing the polynucleotide sequence shown in SEQ ID NO:23). The host bacteria were grown to an optical density of 0.7 at 600 nm in Terrific Broth. Expression of NSP2c was induced by adding IPTG to a final concentration of 1 nm. After incubation for an additional 8-10 hours at 37° C., the bacteria were recovered by centrifugation at 4,200× g for 30 minutes. The cell pellet from a 4L culture was resuspended in 200 ml of lysis buffer (50 mM NaH₂PO₄, 300 mM NaCl, 10 mM imidazole, pH8.0 and 40 μg/ml RNase A) and processed twice using a Microfluidizer high shear processor (Microfluidics, Massachusetts, Model 110Y). To recover His-tagged NSP2c, the clarified lysate was incubated with 8 ml of Ni-NTA agarose beads (QIAGEN™). The beads were washed four times with buffer containing 50 mM NaH₂PO₄, 300 mM NaCl, 20 mM imidazole, pH8.0, and once in a similar buffer containing 50 mM imidazole. Protein was eluted with 70 ml of buffer containing 250 mM imidazole. A final purification was performed using a Centricon centrifugation cartridge. The buffer was changed to 10 mM Tris, 100 mM NaCl, pH 8.0, with a protein concentration of approximately 4-6 mg/ml.

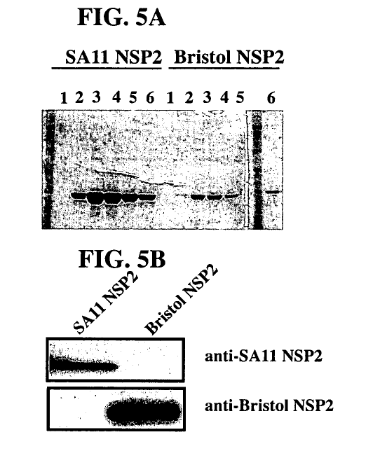

FIG. 5A illustrates the production and recovery of NSP2C octamers as compared to group A (SA11) NSP2 octamers expressed under comparable conditions. Octamers of comparable size and purity were obtained regardless of the group (strain) from which the octamers are derived. These results demonstrate the suitability of group C NSP2 octamers as an epitope mounting platform.

### Example 5

### Presentation of a C-Terminal 19 Peptide NSP1 Fragment by the NSP2 Antigen Delivery Platform

The NSP2-NSP1.C19 fusion protein was used to elicit an immune response. The NSP2:NSP1 fusion protein was prepared by modification of the NSP2 bacterial expression vector pQE60g8 to link the C-terminal 19 amino acids of RRV NSP1 to the C terminus of NSP2 via a GPGP hinge. A His tag was placed downstream of the NSP1 sequence. The purified protein was used to prepare guinea pig polyclonal antisera. (as described in Silvestri et al., J. Virology 78:7763-7774, 2004.

Guinea pigs (4-5 months old) were immunized initially with 0.125 ml (0.2 mg) NSP2-NSP1.C19 fusion protein in Freund's complete adjuvant or without adjuvant. The animals received additional immunizations of the same amount at 2, 4 and 5 weeks, in Freund's incomplete adjuvant or without adjuvant, respectively. FIG. 6 illustrates an exemplary western blot analysis demonstrating the presence of antibodies against the NSP1.C19 antigenic polypeptide in the serum of guinea pigs immunized with an NSP2-NSP1.C19 (SA11) fusion protein.

Cell lysates containing the NSP1 target protein were obtained by infecting MA104 cells with trypsin-activated SA11 strain virus. Proteins were separated by electrophoresis on NuPAGE 10% polyacrylamide gels (InVitrogen) and transferred to nitrocellulose membranes for Western blot analysis. Mock infected cells were utilized as a negative control. Test serum was obtained from guinea pigs immunized as indicated above.

The left panel (+) is serum produced following immunization with the NSP2-NSP1.C19 fusion in adjuvant. The right panel (−) is serum produced following immunization without adjuvant. The center panel is rabbit polyclonal antibody raised against the SA11 C19 peptide (included as a control). Pre- and post-immunization serum lanes are indicated. Mock infected cell lysates (negative control) are shown in lanes “1.” SA11 virus infected cell lysates are shown in lanes “2.” Lanes containing molecular weight standards are indicated by an “M.”

These results show that an NSP1 specific antibodies are produced in response to immunization with an NSP2-NSP1 fusion protein with or without an adjuvant, demonstrating that NSP2 fusion proteins are an effective antigen delivery platform.

### Example 6

### Presentation of Rotavirus VP8 by the NSP2 Antigen Delivery Platform

Guinea pigs (4-5 month old) were immunized with purified 0.25 ml (60 ng) NSP2-VP8 (RRV) fusion protein in low salt buffer (LSB) and equal volume of Freund's complete adjuvant or without adjuvant. The animals received additional immunizations of the same amount at 2, 4, and 5 weeks, in Freund's incomplete adjuvant or without adjuvant, respectively. FIG. 4B depicts a Western blot Analysis of serum obtained from guinea pigs pre- and post-immunization with the NSP2-VP8 fusion protein. Cell lysates from RRV infected cells were separated by electrophoresis on polyacrylamide gels and transferred to nitrocellulose membranes for Western blot Analysis.

FIG. 4B illustrates that guinea pigs immunized with an NSP2-VP8 fusion protein produce antibodies that specifically bind to the VP8 antigenic epitope (VP4). Lane 1 contains post-immunization serum; lane 2 contains pre-immunization serum; lane 3 contains guinea pig hyperimmune sera raised against the DXRRV virus, (included as a control to show the predominant immunoreactive antigens of the virus).

Production of a Serum Antibody Response to the NSP2-VP8 Fusion Protein

Reactivity of serum from immunized guinea pigs was evaluated by sandwich enzyme-linked immunosorbent assay (“ELISA”). In the assay, broadly cross-reactive anti-human rotavirus antisera at a dilution of 1:10,000 was adsorbed on an ELISA plate, followed by sequential immunoadsorption with (1) clarified lysates prepared from rhesus rotavirus (RRV)-infected cells or mock-infected cells, (2) guinea pig serum raised against the NSP2-VP8 fusion protein (at the indicated dilutions), and (3) goat anti-guinea pig antibody conjugated to a horseradish peroxidase at a dilution of 1:1000. The color reaction, developed by adding the TMB substrate (KPL), was read at OD₄₅₀ₙₘ with an ELISA reader.

FIG. 7 illustrates serial 1:4 dilutions of serum from immunized animals incubated with lysates from mock- and RRV-infected cells. Animals immunized with NSP2-VP8 with or without adjuvant produced a specific antibody response which could be detected at greater than 1:500,000 dilution, demonstrating that the antigen delivery platform presented antigen in a manner which elicited a high titer antibody response.

Generation of a Protective Immune Response by the NSP2-VP8 Fusion Protein

Presentation of VP8 using the NSP2 antigen delivery platform elicited a protective antibody response. Serum was obtained from guinea pigs immunized with the NSP2-VP8 antigen delivery platform in adjuvant. Plaque Neutralization Assays were performed by incubating serial dilutions of guinea pig serum with approximately 50 plaque forming units of trypsin activated RRV strain of rotavirus for 1 hour at 37° C. The mixtures were inoculated onto monolayers of MA104 cells contained in multi-well plates, and overlaid with agarose containing 0.5 μg/ml trypsin. Viral plaques were then counted 4 days post-infection. The highest antibody dilution yielding a 50% reduction in plaque formation was designated the neutralization titer. Results of an exemplary plaque neutralization assay are given in Table 1. These results indicated that immunization with the fusion polypeptides described herein elicited a protective immune response against the antigenic polypeptide presented by the antigen delivery platform.

TABLE 1
Plaque Neutralization Assay

| Virus | Anti-sera | N | Neutralization Titer |
|---|---|---|---|
| RRV | Anti-adjNSP2-VP8 | 3 | 640 |
| RRV | preserum | 4 | <10 |

N designates the number of repeats.

Neutralization titer is the highest dilution of the serum showing a 50% reduction of plaque forming units (pfu).

### Example 7

### Lack of Immunological Cross-Reactivity Between Group A and Group C Based Epitope Mounting Platforms

Many individuals are infected with group A rotavirus at some point during early childhood resulting in neutralizing antibodies that react with group A NSP2 protein. To evaluate whether group C derived NSP2 epitope mounting platforms were detected by group A specific antibodies, antibodies specific for either group A (SA11) or group C (Bristol) NSP2 was produced by immunizing guinea pigs with recombinant his-tagged NSP2 octamers. FIG. 5B illustrates a selected portion of a western blot. The top panel shows that antibodies specific for group A rotavirus NSP2 reacted with SA11 strain NSP2 protein, but failed to bind to Bristol strain NSP2 protein. In contrast, antibodies raised against the group C NSP2 protein detected Bristol, but not SA11 protein. This result confirmed that group C NSP2 epitope mounting platforms were not subject to neutralization by serum antibodies specific for group A rotavirus NSP2.

In view of the many possible embodiments to which the principles of this disclosure may be applied, it should be recognized that the illustrated embodiments are only examples of the invention and should not be taken as a limitation on the scope of the invention. Rather, the scope of the invention is defined by the following claims. We therefore claim as our invention all that comes within the scope and spirit of these claims.

We claim:

1. A monomeric fusion protein comprising the following elements linked in an N-terminal to C-terminal direction:
(a) a viral NSP2 polypeptide;
(b) a linear linking peptide; and
(c) an antigenic polypeptide,
wherein a plurality of the monomeric fusion proteins form a self-aggregating multimeric ring structure upon expression.

2. The fusion protein of claim 1, wherein the self-aggregating multimeric ring structure comprises 4, 8, 12 or 16 monomeric fusion proteins.

3. The fusion protein of claim 2, wherein the self-aggregating multimeric ring structure comprises 8 monomeric fusion protein subunits.

4. The fusion protein of claim 1, wherein the viral NSP2 polypeptide comprises a rotavirus NSP2 polypeptide.

5. The fusion protein of claim 1, wherein the viral NSP2 polypeptide is a rotavirus NSP2 polypeptide selected from a Group A rotavirus, a Group B rotavirus, a Group C rotavirus, a Group D rotavirus, a Group E rotavirus, a Group F rotavirus, or a Group G rotavirus.

6. The fusion protein of claim 5, wherein the rotavirus NSP2 polypeptide is a Group A rotavirus NSP2 polypeptide or a Group C rotavirus NSP2 polypeptide.

7. The fusion protein of claim 6, wherein the viral NSP2 polypeptide is a polypeptide with the amino acid sequence of SEQ ID NO:2 or SEQ ID NO:30.

8. The fusion protein of claim 7, wherein the viral NSP2 polypeptide is a polypeptide encoded by a nucleic acid with the polynucleotide sequence of SEQ ID NO:1 or SEQ ID NO:29, or a polynucleotide sequence that differs from SEQ ID NO:1 or SEQ ID NO:29 only by virtue of the degeneracy of the genetic code.

9. The fusion protein of claim 1, the fusion polypeptide comprising at least one affinity tag.

10. The fusion protein of claim 9, the affinity tag comprising a poly-histidine affinity tag.

11. The fusion protein of claim 1, the linear linking peptide comprising at least one amino acid sequence comprising in order the amino acids glycine-proline-glycine-proline or glycine-glycine-serine.

12. The fusion protein of claim 1, the antigenic polypeptide comprising a polypeptide of a pathogenic organism or virus.

13. The fusion protein of claim 1, the antigenic polypeptide comprising a viral or bacterial polypeptide.

14. The fusion protein of claim 13, wherein the antigenic polypeptide comprises a polypeptide of a virus other than a rotavirus.

15. The fusion protein of claim 14, wherein the virus is selected from the group consisting of dengue virus, human immunodeficiency virus, influenza virus, metapneumovirus, norovirus, papillomavirus, parvovirus, SARS virus, smallpox virus, picomaviruses, respiratory syncitial virus, parainfluenza virus, measles, hepatitis, measles, varicella zoster, rabies and West Nile virus.

16. A plurality of fusion proteins of claim 1 assembled into a stable multimeric ring structure.

17. An epitope mounting platform comprising a stable octameric ring structure comprising a plurality of the monomeric fusion proteins of claim 1.

18. An isolated or recombinant nucleic acid encoding a fusion protein, wherein the nucleic acid comprises the following elements in a 5′ to 3′ direction:
(a) a polynucleotide sequence that encodes a viral NSP2 polypeptide;
(b) a polynucleotide sequence that encodes a linear linking peptide; and
(c) a polynucleotide sequence that encodes an antigenic polypeptide.

19. The isolated or recombinant nucleic acid of claim 18, wherein the encoded fusion protein forms a self-aggregating ring structure.

20. The isolated or recombinant nucleic acid of claim 19, wherein the self-aggregating multimeric ring structure comprises 4, 8, 12 or 16 monomeric fusion proteins.

21. The isolated or recombinant nucleic acid of claim 20, wherein the self-aggregating multimeric ring structure comprises 8 monomeric fusion proteins.

22. The isolated or recombinant nucleic acid of claim 18, comprising a rotavirus NSP2 polypeptide.

23. The isolated or recombinant nucleic acid of claim 18, wherein the nucleic acid encodes a viral NSP2 polypeptide that comprises an HIT-like fold and forms a self-aggregating octameric ring structure upon expression.

24. The isolated or recombinant nucleic acid of claim 18, wherein the polynucleotide sequence encoding the viral NSP2 polypeptide comprises the sequence of SEQ ID NO:1, the sequence of SEQ ID NO:29, or a or a polynucleotide sequence that differs from SEQ ID NO:1 or SEQ ID NO:29 only by virtue of the degeneracy of the genetic code.

25. The isolated or recombinant nucleic acid of claim 18, wherein the polynucleotide sequence encoding the linear linking peptide encodes at least one amino acid sequence selected from glycine-proline-glycine-proline and glycine-glycine-serine.

26. The isolated or recombinant nucleic acid of claim 18, further comprising at least one polynucleotide sequence that encodes an affinity tag.

27. The isolated or recombinant nucleic acid of claim 18, wherein the polynucleotide sequence encoding the antigenic polypeptide encodes a polypeptide of a pathogenic organism or virus.

28. The isolated or recombinant nucleic acid of claim 27, wherein the polynucleotide sequence encoding the antigenic polypeptide encodes a viral or bacterial polypeptide.

29. The isolated or recombinant nucleic acid of claim 28, wherein the polynucleotide sequence encoding the antigenic polypeptide encodes a rotavirus polypeptide.

30. The isolated or recombinant nucleic acid of claim 28, wherein the polynucleotide sequence encoding the antigenic polypeptide encodes a polypeptide of a virus other than a rotavirus.

31. The isolated or recombinant nucleic acid of claim 30, wherein the virus is selected from the group consisting of dengue virus, human immunodeficiency virus, influenza virus, metapneumovirus, norovirus, papillomavirus, parvovirus, SARS virus, smallpox virus, picornaviruses, respiratory syncitial virus, parainfluenza virus, measles, hepatitis, measles, varicella zoster, rabies and West Nile virus.

32. A vector comprising the polynucleotide of claim 18.

33. An immunogenic composition comprising at least one of:
(a) a multimeric fusion protein ring structure comprising a plurality of monomeric fusion proteins; and
(b) a recombinant polynucleotide encoding a monomeric fusion protein, wherein a plurality of monomeric fusion proteins form a self-aggregating multimeric ring structure;
each of the monomeric fusion proteins comprising the following elements linked in an N-terminal to C-terminal direction:
(i) a viral NSP2 polypeptide;
(ii) a linear linking peptide; and,
(iii) an antigenic polypeptide.

34. The immunogenic composition of claim 33, wherein the self-aggregating multimeric ring structure comprises 4, 8, 12 or 16 monomeric fusion proteins.

35. The immunogenic composition of claim 34, wherein the self-aggregating multimeric ring structure comprises 8 monomeric fusion proteins.

36. The immunogenic composition of claim 33, comprising a rotavirus NSP2 polypeptide.

37. The immunogenic composition of claim 36, wherein the viral NSP2 polypeptide is a polypeptide with the amino acid sequence of SEQ ID NO:2 or SEQ ID NO:30.

38. The immunogenic composition of claim 33, the antigenic polypeptide comprising a polypeptide of a pathogenic organism or virus.

39. The immunogenic composition of claim 33, the antigenic polypeptide comprising a viral or bacterial polypeptide.

40. The immunogenic composition of claim 39, wherein the antigenic polypeptide comprises a rotavirus polypeptide.

41. The immunogenic composition of claim 39, wherein the antigenic polypeptide comprises a polypeptide of a virus other than a rotavirus.

42. The immunogenic composition of claim 41, wherein the virus is selected from the group consisting of dengue virus, human immunodeficiency virus, influenza virus, metapneumovirus, norovirus, papillomavirus, parvovirus, SARS virus, smallpox virus, picomaviruses, respiratory syncitial virus, parainfluenza virus, measles, hepatitis, measles, varicella zoster, rabies and West Nile virus.

43. The immunogenic composition of claim 33, wherein the immunogenic composition is a vaccine.

44. A pharmaceutical composition comprising at least one of:
(a) a multimeric fusion protein ring structure comprising a plurality of monomeric fusion proteins; and
(b) a recombinant polynucleotide encoding a monomeric fusion protein, wherein a plurality of monomeric fusion proteins form a self-aggregating multimeric ring structure;
each of the monomeric fusion proteins comprising the following elements linked in an N-terminal to C-terminal direction:
(i) a viral NSP2 polypeptide;
(ii) a linear linking peptide; and
(iii) an antigenic polypeptide;
and a pharmaceutically acceptable carrier or excipient.

45. The pharmaceutical composition of claim 44, wherein the carrier or excipient further comprises one or more of aluminum hydroxylphosphosulfate, alum, CRM₁₉₇ and liposomes.

46. A method of generating an immune response against an antigenic polypeptide, the method comprising:
administering to a mammal at least one of:
(a) a multimeric fusion protein ring structure comprising a plurality of monomeric fusion proteins; and
(b) a recombinant polynucleotide encoding a monomeric fusion protein comprising the following components linked in an N-terminal to C-terminal direction:
(i) a viral NSP2 polypeptide;
(ii) a linear linking peptide; and,
(iii) an antigenic polypeptide,
wherein a plurality of monomeric fusion proteins form a self-aggregating multimeric ring structure upon expression.

47. The method of claim 46, wherein the immune response is generated by:
(a) expressing in a cell a recombinant polynucleotide encoding a monomeric fusion protein comprising the following components linked in an N-terminal to C-terminal direction:
(i) a viral NSP2 polypeptide;
(ii) a linear linking peptide; and
(iii) an antigenic polypeptide,
wherein a plurality of monomeric fusion proteins form a plurality of self-aggregating multimeric ring structures;
(b) recovering the multimeric ring structures from the cell; and
(c) administering the multimeric ring structures to a mammal.

48. The method of claim 47, wherein the multimeric ring structures are recovered by affinity chromatography.

49. The method of claim 46, wherein the multimeric ring structures comprise a plurality of antigenic polypeptide, thereby generating an immune response to a plurality of antigenic polypeptides.

50. The method of claim 46, wherein the antigenic polypeptide is a polypeptide of a pathogenic or virus.

51. The method of claim 46, wherein the pathogenic organism is a bacterium.

52. The method of claim 46, wherein the virus is a rotavirus.

53. The method of claim 46, wherein the virus is a virus other than a rotavirus.

54. The method of claim 53, wherein the virus is selected from the group consisting of dengue virus, human immunodeficiency virus, influenza virus, metapneumovirus, norovirus, papillomavirus, parvovirus, SARS virus, smallpox virus, picornaviruses, respiratory syncitial virus, parainfluenza virus, measles, hepatitis, measles, varicella zoster, rabies and West Nile virus.

55. The method of claim 46, wherein the polynucleotide encoding the monomeric fusion protein comprises a recombinant non-viral plasmid vector or a recombinant plasmid viral vector.

56. The method of claim 46, wherein the monomeric fusion protein further comprises at least one affinity tag.

57. The method of claim 56, wherein the monomeric fusion protein comprises at least one six-histidine affinity tag.

58. The method of claim 56, wherein the multimeric ring structures are recovered by affinity chromatography with an affinity resin comprising nickel, cobalt or a combination of nickel and cobalt.

59. A method of producing a recombinant immunogen by expressing in a cell a monomeric fusion protein comprising the following components linked in an N-terminal to C-terminal direction:
(i) a viral NSP2 polypeptide;
(ii) a linear linking peptide; and
(iii) an antigenic polypeptide,
wherein a plurality of monomeric fusion proteins form a self-aggregating multimeric ring structure.

60. The method of claim 59, comprising expressing the monomeric fusion protein in a bacterial cell.

61. The method of claim 59, comprising expressing the monomeric fusion protein in a eukaryotic cell.

62. The method of claim 59, comprising expressing the monomeric fusion protein by introducing a polynucleotide encoding the monomeric fusion protein into a cell.

63. A self-aggregating monomeric fusion protein made by the method of claim 59.
